(19) United States
(12) Patent Application Publication
(10) Pub. No.: US 2022/0034911 A1
(43) Pub. Date: Feb. 3, 2022

(54) METHOD OF MITIGATION OF DEATH FROM EPILEPTIC SEIZURES

(71) Applicant: Jose VEGA, Miami, FL (US)

(72) Inventor: Jose VEGA, Miami, FL (US)

(21) Appl. No.: 16/965,994

(22) PCT Filed: Sep. 9, 2019
(86) PCT No.: PCT/US2019/050242

§ 371 Date: Jul. 29, 2020

Related U.S. Application Data
(60) Provisional application No. 62/729,159, filed on Sep. 10, 2018.
(60) Provisional application No. 62/732,854, filed on Sep. 18, 2018.
(60) Provisional application No. 62/831,479, filed on Apr. 9, 2019.

(51) Int. Cl. G01N 33/68; A61B 10/00; A61B 5/024; A61B 5/00

(52) U.S. Cl. CPC G01N 33/6896; A61B 10/0051; G01N 2800/2857; A61B 5/4094; A61B 5/024

(57) ABSTRACT

A method for determining the severity of a mammalian dive response (MDR) triggered during a patient's seizure that includes the following steps: (1) collecting an ictal specimen, the ictal specimen being a saliva or any other secretion from the mouth of the patient when the patient is in a peri-ictal period; (2) analyzing the ictal specimen to measure a level of a specimen marker, the specimen marker being a cellular or non-cellular biological component; and (3) determining the severity of the MDR or other autonomic reflex based on the level of the specimen marker.

This application is the national stage application of PCT/US2019/050242, filed on Sep. 9, 2019, which claims the benefit of U.S. Provisional Application No. 62/729,159, filed on Sep. 10, 2018; 62/732,854, filed on Sep. 18, 2018; and 62/831,479, filed on Apr. 9, 2019, all of which are incorporated by reference for all purposes as if fully set forth herein.

### FIELD OF THE INVENTION

The present invention relates to methods and kits to assess the risk of sudden death due to epileptic seizures, and the use of a pharmacological approach to prevent such a death.

### BACKGROUND OF THE INVENTION

Sudden death due to epileptic seizures is common. Such a death occurs most frequently in the setting of (1) abrupt cessation of breathing, (2) severe slowing of the heart rate, (3) pulmonary edema or hemorrhage, and (4) the results of severely elevated systemic blood pressure.

To date, the investigation of sudden death from epileptic seizures has been a challenge because most seizure-related deaths are sudden and unexpected, which prevents scientists from making direct observations during death-inducing seizures. Therefore, there is currently no reliable way to predict which patients are at risk of death from epileptic seizures, and even if there was, there is no way to prevent it.

### SUMMARY OF THE INVENTION

In one embodiment, the present invention provides a method for determining the severity of a mammalian dive response (MDR) or other autonomic reflex triggered during a seizure. The method includes the following steps: (1) collecting an ictal specimen, the ictal specimen being a saliva or any other secretion which pools in or around the mouth of a patient during a seizure; (2) analyzing the ictal specimen to measure a level of a specimen marker, the specimen marker being a cellular or non-cellular biological component; and (3) determining the severity of the MDR or other autonomic reflex based on the level of the specimen marker.

In another embodiment, step (2) includes: collecting a reference specimen, the reference specimen being a saliva or any other secretion from the mouth of the patient when the patient is not having a seizure, or from a pool of healthy age-appropriate seizure-free volunteers; and analyzing the ictal specimen to measure the level of the specimen marker; analyzing the reference specimen to measure a level of the specimen marker; and comparing the level of the specimen marker in the ictal specimen with the level of the specimen marker in the reference specimen.

In another embodiment, the ictal specimen and the reference specimen are a tissue, a fluid, a cell, an electrolyte, or a molecule found in or around a respiratory system.

In another embodiment, the ictal specimen and reference specimen are a cell that makes up an oro-nasal cavity, a pharyngeal cavity, or a lung parenchyma; a cell that makes up lung blood vessels; an electrolyte; a molecule that makes up connective tissues of lungs and tracheobronchial tree; a cells or platelet that circulates throughout lung, peritracheal, peribronchial arteries, veins or lymphatic vessels; or a molecule, cell or fluid that makes up or that extravasates into lung alveoli, or into the lumen of tracheobronchial tree.

In another embodiment, the specimen marker is a biological component that enters the lungs through pulmonary arteries.

In another embodiment, the specimen marker is selected from the group consisting of electrolytes, carbohydrates, proteins, lipids, and nucleic acids.

In another embodiment, the specimen marker is a surfactant.

In another embodiment, step (3) includes: determining that the severity of the MDR or other autonomic reflex is high and a prophylactic pharmacological approach is required when the level of the specimen marker in the ictal specimen is 2 times, 5 times, or 10 times higher than the level of the specimen marker in the reference specimen.

In another embodiment, the prophylactic pharmacological approach is to administer to the patient an adrenergic blocking agent selected from the group consisting of n-g(fluorenyl)-n ethyl-β chloroethylamine (SKF-501), Phentolamine, Doxazosin, Terazosin, Tamsulosin, Alfuzosin, Phenoxybenzaamine, Yohimbine, Imipramine, Silodosin, Amitriptilyne, Indoramine, Abanoquil, Corynanthine, Ajmalicine, Quetiapine, Dapiprazone, Amoxapine, Risperidone, Idazoxan, Bunazosin, Doxepin, Atiprosin, Piperoxan, Fenmetozole, Denopamine, Dobutamine, Dopexamine, Epinephrine, Isoprenaline, Isoproterenol, Prenalterol, Xamotero, Arformoterol, Buphenine, Clenbuterol, Dopexamine, Fenoterol, Formoterol, Isoetarine, Levosalbutamol, levalbuterol, Orciprenaline, metaproterenol, Pirbuterol, Procaterol, Ritodrine, Salbutamol, albuterol, Salmeterol, Terbutaline, Arbutamine, Befunolol, Bromoacetylalprenololmenthane, Broxaterol, Cimaterol, Cirazoline, Etilefrine, Hexoprenaline, Higenamine, Isoxsuprine, Mabuterol, Methoxyphenamine, Oxyfedrine, Ractopamine, Reproterol, Rimiterol, Tretoquinol, Tulobuterol, Zilpaterol, Zinterol, Propranolol, Bucindolol, Carteolol, Carvedilol, Labetalol, Nadolol, Oxprenolol, Penbutolol, Pindolol, Sotalol, Timolol, Acebutolol, Atenolol, Betaxolol, Bisoprolol, Celiprolol, Metoprolol, Nebivolol, Esmolol, Butaxamine, ICI-118,551, SR 59230A, 4-NEMD, 7-Me-marsanidine, Agmatine, Apraclonidine, Brimonidine, Cannabigerol, Clonidine, Detomidine, Dexmedetomidine, Fadolmidine, Guanabenz, Guanfacine, Lofexidine, Marsanidine, Medetomidine, Methamphetamine, Mivazerol, Rilmenidine, Romifidine, Talipexole, Tiamenidine, Tizanidine, Tolonidine, Xylazine, Xylometazoline, L-748,328, L-748,337, SR 59,230A, Amibegron (SR-58611A), CL-316,243, L-742,791, L-796,568, LY-368,842, Mirabegron (YM-178), Ro40-2148, Solabegron (GW-427,353), and Vibegron (MK-4618); or to administer to the patient a sympathetic ganglionic blocking agent selected from the group consisting of hexamethonium, pentolinium, mecamylamine, trimetaphan, tubocurarine, pempidine, benzohexonium, chlorisondamine, pentamine, and tetra ethylammonium chloride; or to administer to the patient a cholinergic receptor blocking agent selected from the group consisting of atropine, scopolamine, homatropine, tropicamide, benzproine, biperiden, trihexyphenidyl, oxybutynin, tolterodine, solifenacin, dicyclomine, darifenacin, glycopyrrolate, ipratropium, and tiotropium; to administer to the patient a combination thereof.

In another embodiment, step (2) includes an analysis selected from a group consisting of mass spectrometry, protein profile analysis, protein chip array analysis, lipid profile analysis, nucleic acid profile analysis, electrolyte analysis, and immunofluorescence.

In another embodiment, the ictal specimen and the reference specimen are measured by a portable sample analyzing unit.

In one embodiment, the present application provides a method for determining the severity of a mammalian dive response (MDR) or other autonomic reflex triggered during a seizure. The method includes the following steps: (1) collecting an ictal blood sample from a patient when the patient is in a peri-ictal period; (2) analyzing the ictal blood sample for the levels of one or more blood markers; (3) collecting a reference blood sample from the patient when the patient is not having a seizure; (4) analyzing the reference blood sample for the levels of the one or more blood markers; and (5) comparing the levels of the one or more blood markers in the ictal blood sample and the levels of the one or more blood markers in the reference blood sample.

In another embodiment, the one or more blood markers are selected from the group consisting of electrolytes, carbohydrates, proteins, lipids, nucleic acids, cell surface molecules originating in a human body tissue (e.g., lungs, muscles, liver, spleen, intestines, bone marrow, arteries, veins, etc.), white blood cells, red blood cells, platelets, pH, lactic acid level, carbon dioxide level, oxygen level, and blood oxygen saturation level.

In another embodiment, the method further includes: measuring the patient's pulse.

In another embodiment, the method further includes: determining that the severity of the MDR or other autonomic reflex triggered during a seizure is severe and a prophylactic pharmacological approach is required when the levels of the one or more blood markers in the ictal blood sample are 2 times, 5 times, or 10 times than the levels of the one or more blood markers in the reference blood sample.

In another embodiment, the method further includes: determining that the severity of the MDR or other autonomic reflex triggered during a seizure is severe and a prophylactic pharmacological approach is required when the levels of the one or more blood markers in the ictal blood sample are 3%, 5%, or 10% lower than the levels of the one or more blood markers in the reference blood sample, wherein the one or more blood markers are pH or oxygen level.

In another embodiment, the method further includes: determining that the severity of the MDR or other autonomic reflex triggered during a seizure is high and a prophylactic pharmacological approach is required when the white blood cell count in the ictal blood sample is higher than 9,200 cells/mm³, 16,000 cells/mm³, 18,000 cells/mm³, 20,000 cells/mm³, 22,000 cells/mm³, 24,000 cells/mm³, 26,000 cells/mm³, 28,000 cells/mm³, 30,000 cells/mm³, or 32,000 cells/mm³, wherein the one or more blood markers are white blood cells.

In another embodiment, the ictal blood sample and reference blood sample are measured by a portable sample analyzing unit.

In another embodiment, the prophylactic pharmacological approach is to administer to the patient an adrenergic blocking agent selected from the group consisting of n-g(fluorenyl)-n ethyl-β chloroethylamine (SKF-501), Phentolamine, Doxazosin, Terazosin, Tamsulosin, Alfuzosin, Phenoxybenzaamine, Yohimbine, Imipramine, Silodosin, Amitriptilyne, Indoramine, Abanoquil, Corynanthine, Ajmalicine, Quetiapine, Dapiprazone, Amoxapine, Risperidone, Idazoxan, Bunazosin, Doxepin, Atiprosin, Piperoxan, Fenmetozole, Denopamine, Dobutamine, Dopexamine, Epinephrine, Isoprenaline, Isoproterenol, Prenalterol, Xamotero, Arformoterol, Buphenine, Clenbuterol, Dopexamine, Fenoterol, Formoterol, Isoetarine, Levosalbutamol, levalbuterol, Orciprenaline, metaproterenol, Pirbuterol, Procaterol, Ritodrine, Salbutamol, albuterol, Salmeterol, Terbutaline, Arbutamine, Befunolol, Bromoacetylalprenololmenthane, Broxaterol, Cimaterol, Cirazoline, Etilefrine, Hexoprenaline, Higenamine, Isoxsuprine, Mabuterol, Methoxyphenamine, Oxyfedrine, Ractopamine, Reproterol, Rimiterol, Tretoquinol, Tulobuterol, Zilpaterol, Zinterol, Propranolol, Bucindolol, Carteolol, Carvedilol, Labetalol, Nadolol, Oxprenolol, Penbutolol, Pindolol, Sotalol, Timolol, Acebutolol, Atenolol, Betaxolol, Bisoprolol, Celiprolol, Metoprolol, Nebivolol, Esmolol, Butaxamine, ICI-118,551, SR 59230A, 4-NEMD, 7-Me-marsanidine, Agmatine, Apraclonidine, Brimonidine, Cannabigerol, Clonidine, Detomidine, Dexmedetomidine, Fadolmidine, Guanabenz, Guanfacine, Lofexidine, Marsanidine, Medetomidine, Methamphetamine, Mivazerol, Rilmenidine, Romifidine, Talipexole, Tiamenidine, Tizanidine, Tolonidine, Xylazine, Xylometazoline, L-748,328, L-748,337 and SR 59,230A, Amibegron (SR-58611A), CL-316,243, L-742,791, L-796,568, LY-368,842, Mirabegron (YM-178), Ro40-2148, Solabegron (GW-427,353), and Vibegron (MK-4618); or to administer to the patient a sympathetic ganglionic blocking agent selected from the group consisting of hexamethonium, pentolinium, mecamylamine, trimetaphan, tubocurarine, pempidine, benzohexonium, chlorisondamine, pentamine, and tetra ethylammonium chloride; or to administer to the patient a cholinergic receptor blocking agent selected from the group consisting of atropine, scopolamine, homatropine, tropicamide, benzproine, biperiden, trihexyphenidyl, oxybutynin, tolterodine, solifenacin, dicyclomine, darifenacin, glycopyrrolate, ipratropium, and tiotropium; or to administer to the patient a combination thereof.

In one embodiment, the present invention provides a method for treating epileptic seizures. The method includes administering to a patient a combination of an adrenergic blocking agent and an anti-seizure agent; or a combination of a cholinergic blocking and an anti-seizure agent.

In another embodiment, the anti-seizure agent is selected from the group consisting of phenytoin, carbamazepine, clobazam, divalproex, eslicarbazepine, oxcarbazepine, lacosamide, ethosuximide, gabapentin, methsuxamide, perampanel, phenobarbital, pregabalin, rufinamide, tiagabine, vigabatrin, levetiracetam, clonazepam, clorazepate, ezogabine, felbamate, lamotrigine, lorazepam, primidone, topiramate, valproic acid, and zonisamide.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 shows WBC count dynamics in the presence and absence of PeCRC. A. PoCL occurred most frequently in patients who experienced PeCRC (PeCRC+), and only rarely in patients who did not experience PeCRC (PeCRC−) (OR=17.0; 95% CI 7.2-40.9; p<0.0001). B. PeCRC+ patients most frequently demonstrated WBC1 counts that fell within the upper two quintiles, while PeCRC− patients most frequently demonstrated WBC1 counts that fell in the two lowest quintiles of the WBC count range (3,300-31,200 cells/mm³; p<0.001; Chi square test for trend). C. Initial WBC count elevations measured in the emergency department experienced a significant decrease within the first 24 h. Such elevations occurred beyond the normal WBC range for the PeCRC+ group of patients, but within the normal range for the PeCRC− group. Error bars represent the mean±s.e.m. ***p<0.001. D. The RLI was positive in the groups shown at much higher rates than expected by chance, including patients who did not experience PoCL (PoCL−). Notably, the highest frequency of positive RLIs was seen in the PeCRC+ group. The percentages written above the top panels represent the observed frequency with which the RLI was positive for each group; the p values show the significance of a binomial test comparing each observed frequency with an expected frequency of 50%. E-G. Comparison of normalized WBC count and HCT changes from CBC1 to a blood count CBC2. The p values above each graph show the significance of within group paired comparisons using a two-tailed Wilcoxon matched pairs sign-ranked test. Error bars show the mean±s.e.m. Comparisons specifically relevant to the entire cohort, to PoCL− patients, to patients who did not require endotracheal intubation (INT−) and to patients who required endotracheal intubation (INT+) can be found in FIG. 4. CBC=Complete blood count; CBC1=CBC performed within 12 hours of arrival at the emergency department (CBC1); CBC2=CBC performed within 36 hours of CBC1; HCT=hematocrit; PeCRC=Periconvulsive respiratory compromise; PoCL=Postconvulsive leucocytosis; RLI=Relative leucocytosis index; WBC=white blood cell. WBC1=Initial WBC count measured in the emergency department.

FIG. 2 shows the relationship between PeCRC, WBC1 count, heart rate, and endotracheal intubation. A-B. PeCRC+ and PoCL+ patients exhibited increased heart rates compared with their PeCRC− and PoCL− counterparts. Error bars show mean±s.e.m. C. A correlation between heart rate and WBC1 count is evident in PeCRC+ patients (n=48; pink circles) and is particularly strong among PeCRC+ INT+ patients (n=21; blue circles). Note that this correlation is no longer present when tested in the entire patient cohort (n=152; orange circles). P values represent significance of goodness of fit data obtained via linear regression. D. PeCRC+ patients exhibit acute CXR abnormalities more frequently than PeCRC− patients (p<0.003; 95% CI=1.5-8.0; Fishers exact test; also see Table 1). E. The mean WBC1 count of PeCRC+ INT+ patients significantly exceeded that of PeCRC+ INT− patients. Note that all PeCRC+ patients whose WBC count exceeded 18,000 cells/mm³ required intubation. *p<0.05; Error bars show mean±s.e.m. F. AUC obtained from a ROC analysis of WBC1 and heart rate performance as biomarkers for endotracheal intubation (i.e., respiratory collapse). The performance of WBC1 is particularly strong, while, while still significant, that of heart rate is modest. Specific ROC cutoff values for WBC1 and heart rate are shown in the Tables 8 and 9, respectively. AUC=Area under the curve; CBC=Complete blood count; CXR=Chest X-ray; HCT=hematocrit; INT+=patients who required intubation; INT-=patients who did not require intubation; PeCRC=Periconvulsive respiratory compromise; PoCL=Postconvulsive leucocytosis; ROC=receiving operator characteristic; RLI=Relative leucocytosis index; WBC=white blood cell. WBC1=initial WBC count measured in the emergency department.

FIG. 3 shows the fraction of patients in each group that demonstrated triage SaO2<95%. PeCRC+=experienced peri-ictal respiratory compromise. PeCRC−=did not experienced peri-ictal respiratory compromise. SaO2=oxygen saturation measured by pulse oximetry at ED triage.

FIG. 4 shows normalized postconvulsive changes in WBC count and HCT. Comparison of percent normalized WBC count and hematocrit changes from complete blood counts (CBC) performed within 12 hours of arrival at the emergency department and CBCs performed within the following 36 hours; The p values above each graph show the significance of paired comparisons using a two-tailed Wilcoxon matched pairs sign-ranked test. Error bars show the mean±s.e.m. Graphs for PoCL−, PeCRC− and PeCRC+ groups can be found in FIG. 1E-G, respectively. All=Full cohort; HCT=Hematocrit; INT+=Group of patients who required emergent endotracheal intubation; INT-=group of patients who did not require endotracheal intubation; PoCL+=Group of patients who experienced postconvulsive leucocytosis; WBC=white blood cell count.

FIG. 5 shows convulsions accompanied by severe respiratory compromise induce a concurrent increase heart rate, lactic acid and platelets. A-B: Patients who experienced PeCRC and who required emergent endotracheal intubation (PeCRC+ INT+) experienced a simultaneous increase in WBC count and in lactic acid. This simultaneous increase was not observed in GCS patients who demonstrated PeCRC but who did not require emergent endotracheal intubation (PeCRC+ INT−). C: GCS patients who require emergent endotracheal intubation (INT+) also demonstrate higher post-convulsive platelet levels than patients who do not require emergent endotracheal intubation (INT−). PeCRC=peri-ictal respiratory compromise; GCS=generalized convulsive seizures.

### DETAILED DESCRIPTION OF THE ILLUSTRATED EMBODIMENTS

Reference will now be made in detail to embodiments of the present invention.

In the present application, the following terms are defined as followings. Adrenergic blocking agent: any medication that decreases the effects of epinephrine and norepinephrine on adrenergic receptors which mediate vasoconstriction, tachycardia, and other typical functions of the sympathetic nervous system. Blood marker: Any measurable electrolyte, cellular or non-cellular biological component found in an ictal blood sample. Generalized convulsive seizure: An epileptic seizure. Herein this term is used interchangeably with the terms “seizure,” “epileptic seizure”, and “epileptic convulsion”. Ictal blood sample: a blood sample collected from a patient via a plurality of methods immediately after the end of a seizure of interest. Ictal Specimen: a saliva or any other secretion which pools in or around the mouth during a seizure. Mammalian dive response: a set of autonomic reflexes triggered by prolonged apnea. Periconvulsive respiratory compromise: Any sign of respiratory compromise during or immediately after a convulsive seizure. Peri-ictal period: time lapse starting at the onset of an epileptic seizure and ending after the patient returns to his or her normal seizure-free baseline state. This period typically last several minutes, but it can last several hours. Postconvulsive leucocytosis: Elevation of the white blood cell count beyond the normal reference range, immediately after a convulsion. Relative leucocytosis index. An indirect measure of whether the white blood cell count measured after a convulsive seizure represents a relative elevation from its pre-seizure baseline. Relative postconvulsive leucocytosis: a relative elevation of the white blood cell count in the peripheral blood occurring immediately after a convulsive seizure. This elevation can remain within, or exceed, the normal white blood cell count reference range. Reference blood sample: a blood sample collected via a plurality of methods from a patient of interest who has not had a seizure in the recent past. Reference blood samples can also be obtained from healthy, age-appropriate volunteers who do not suffer from seizures. Reference specimen: A saliva or any other secretion which can be collected from the mouth, nose, or oropharyngeal cavity of a healthy person who is not having a seizure. Reference specimens can be obtained from a seizure patient of interest, or from healthy, age-appropriate seizure-free volunteers. Specimen marker: Any measurable cellular or non-cellular biological component found in an ictal specimen. Sympathetic ganglionic blocking agent: any medication that decreases or inhibits the release of epinephrine and norepinephrine from postganglionic sympathetic neuronal terminals, thereby decreasing vasoconstriction, tachycardia, and other typical functions of the sympathetic nervous system. White blood cell count: Number of white blood cells (i.e., leucocytes) per mm³ found in a peripheral blood sample.

In a recent article [Vega J L (2018) Ictal Mammalian Dive Response: A Likely Cause of Sudden Unexpected Death in Epilepsy. Front. Neurol. 9:677. doi: 10.3389/fneur.2018.00677], this inventor presented evidence which strongly suggests that sudden death from a seizure is caused by the pathophysiological results of a set of neurological reflexes known as the mammalian dive response (MDR). In humans, the MDR is unleashed by either a voluntary or an involuntary prolonged cessation of breathing (i.e., prolonged apnea). The MDR induced by voluntary cessation of breathing is mild and is terminated voluntarily. By contrast, seizures often induce prolonged apnea, in combination with high metabolic demands from vigorous convulsions which induces a particularly severe form of the MDR, which cannot be controlled voluntarily, and that results in the following physiological events:

(1) diving bradycardia or severe slowing of the heart rate

(2) severe constriction of the majority of the body's blood vessels; and/or

(3) contractions of the spleen.

Each of these physiological events leaves one or more detectable transient biological clues that can help determine if a severe and potentially harmful form of the MDR, or possibly other autonomic reflex, was triggered. If these transient biological clues are found after one or more seizures, it can be assumed that the patient who has suffered these seizures will likely continue to experience severe forms of the MDR during his or her future seizures because the large majority of patients exhibit consistent seizure phenomenology.

Patients who experience the most severe forms of these autonomic reflexes could die from two main conditions: (1) terminal diving bradycardia and (2) terminal hypoxia, probably from pulmonary edema. Even when they are not lethal, the physiological events associated with these conditions can be detected in the bloodstream. These include low oxygen gas level, low oxygen hemoglobin saturation, low pH, high lactic acid, high carbon dioxide, and either a very high, or a very low heart rate. These events and physiological changes can be detected using a plurality of standard methods that include pulse oximetry, capillary, arterial, or venous blood analysis.

In addition, the peripheral vasoconstriction of the MDR can lead to increases in the amount of fluid being transported through the pulmonary arteries, veins and lymphatic vessels, which not only can cause pulmonary edema, but can also induce the formation of a frothy and foamy fluid in some of the lung tissues.

This foamy fluid contains lung-derived proteins and cells that can be recovered from various places in the respiratory system, including but not limited to the nose and mouth. These lung-derived proteins can be identified from a sample of this foamy fluid collected and used for immediate analysis, or collected and preserved for future analysis using a plurality of assays or tests, examples of which include but are not limited to, mass chromatography and different types of immonoassays, among other tests. In addition, the same physiological events that cause pulmonary edema can release red and white blood cells, as well as platelets and other cellular and non-cellular components from the lungs, spleen and other organs. These components can also be recovered from the bloodstream in the peri-ictal period, and be preserved after collection for subsequent detection at a laboratory, using standard cellular and non-cellular detection techniques such as HPLC, immunofluorescence, ELISA, flow cytometry, among many others, or using a strategic portable sample analyzing unit.

The MDR can be attenuated by adrenergic blocking agents and by cholinergic blocking agents.

This invention consists of a system that uses one or more of the measurable physiological correlates of the MDR, to identify whether a patient's seizures place him or her at risk of sudden unexpected death. It involves two test kits which can be used independently of each other, or together, to predict the relative risk of seizure-induced sudden death. It is based on the premise that the severity of an MDR triggered during a seizure is directly proportional to the risk of terminal bradycardia, pulmonary edema, respiratory failure and death from that seizure.

Consequently, the quantification of physiological markers left behind by the MDR triggered during a seizure can be used to predict the risk of sudden death from a seizure. The test kits described below can be used by patients and/or their families, or other bystanders, immediately after the end of any of seizure of interest. The results from this test kit can help decide whether a patient will require the immediate or chronic use of a prophylactic medication to lower the risk of death from his or her seizure.

The first test kit measures specimen markers from the ictal specimen and reference specimen.

Determination of risk of death from seizure using this first test kit, can be obtained using one of two methods. The first method involves obtaining pooled average normal levels of specimen markers found in the saliva of age-appropriate healthy volunteers. Deviations from these normal levels found in ictal specimen of a given seizure patient will help determine his or her specific risk of death from a future seizure. The greater the deviation from these normal levels, the greater the risk of death. For a hypothetical example, under normal physiological conditions surfactant is a specimen marker that is found at high concentrations inside the lung alveoli but at very low or undetectable concentrations in secretions collected from the mouth of normal healthy people (i.e., reference specimen). Following a dangerous seizure which affects respiration, the concentration of surfactant is likely to increase in secretions collected from the mouth (i.e., ictal specimen). The detection of this increased concentration of surfactant signals that the patient has suffered a dangerous seizure, which has generated a certain degree of pulmonary pathology which has the potential to affect respiration, and to cause death. Thus, if the average level of surfactant in the oral secretions of a pool of non-epileptic healthy volunteers is 0.1 mg/ml, but after a seizure a given patient's surfactant concentration in the ictal specimen increases to 1.0 mg/ml, this 10-fold increase can be used to quantify the relative risk that his seizures can cause him to die from respiratory problems during a subsequent seizure.

The second method to determine risk of death from a seizure involves comparing the ictal specimen with a reference specimen collected from the same patient. This ensures that the specimen markers tested by the kit are at their normal, baseline levels. The greater the difference between the levels of relevant specimen markers found between the ictal specimen and the reference specimen, the greater the patient's risk of death. Therefore, as an example if the patient's reference specimen is 0.1 mg/ml of a specimen marker but increases to 1.0 mg/ml in the ictal specimen, this 10-fold increase can be used to quantify the relative risk that his seizures can cause him to die from respiratory problems during a subsequent seizure.

The second test kit uses blood collected from the patient by the patient himself, by any bystander, or by any medical professional, during or immediately after the end of a seizure of interest. This second test kit combines the quantification of various specimen markers and body markers (combined as blood markers therein) of MDR deployment, including (1) number estimates, mass estimates, chemical composition, or specific cell markers (e.g., any electrolyte, any organ or any cell-specific protein, lipid, or nucleic acid of tissue cells, white blood cells, red blood cells or platelets), (2) pH, (3) lactic acid level, (3) CO₂ level, and (4) O₂ level and (5) a measurement of hemoglobin oxygen saturation which can be obtained by a plurality of methods. For a hypothetical example, lactic acid is a small molecule that is normally found at very low concentrations in the bloodstream of healthy patients, but which can increase following the kind of seizures that can result in death. The average concentration of lactic acid in the normal blood is 0.5-1.0 mmol/L but increases several fold following seizures, especially if such seizures affect breathing (see attached paper for evidence). Consequently, the detection of increased blood lactic acid level signals that the patient has suffered a dangerous seizure, which affects respiration, and thus has the potential to cause death. Thus, if the level of lactic acid found in the ictal blood sample increases to 10 mmol/L from its normal baseline level in a reference blood sample, this 10-fold increase can be used to quantify the relative risk that this patient's subsequent seizures can cause him to die. Similar comparisons of other specimen markers and body markers between the reference blood sample and the ictal blood sample can be used to determine the risk of respiratory failure and possible death during a seizure (see attached paper for evidence).

Determination of risk of death from a seizure using this second kit can be obtained using one of two methods. The first method involves obtaining the reference blood sample from age-appropriate healthy volunteers. Deviations from the average control levels of relevant specimen and body markers will determine the risk of death during a seizure. The greater the deviation from these control levels, the greater the risk of death (see example above).

The second method to determine risk of death from seizures using this second kit involves comparing the patients' ictal blood sample with a reference blood sample obtained from his own blood at time when he/she is seizure-free.

In one embodiment of this invention any, all, or any combination of the results from either the first kit or the second kit, or the pulse rate, or the hemoglobin oxygen saturation obtained via a portable pulse oximeter provided to the patient, can be used independently of all other results in order to calculate the risk of death from the patient's seizures. In another embodiment of this invention any, all, or any combination of the results from the first test kit, can be used in combination with any, all, or any combination of the results from the second kit, or with the pulse rate, or with the pulse oxygen saturation, to assess risk of death from a seizure.

The results obtained from the test kits are used to determine which patients will benefit from the immediate or the chronic use of prophylactic medication or medications aimed to attenuate or prevent the physiological consequences of severe forms of the MDR triggered by a seizure. Even though in the present application the risk of death from a seizure is to be used in conjunction with a medication given to prevent such a death, it should be clear that such a medication can be used independently of the test kits presented herein (i.e., the medication can be useful even if methods of risk assessment not described in this application are used).

Any adrenergic blocking agents, any sympathetic ganglionic blockers, or any combination of these agents (denominated adrenergic blocking agents from here on), including but not limited to compounds listed in the following list can be used for this purpose:

n-g(fluorenyl)-n ethyl-β chloroethylamine (SKF-501), Phentolamine, Doxazosin, Terazosin, Tamsulosin, Alfuzosin, Phenoxybenzaamine, Yohimbine, Imipramine, Silodosin, Amitriptilyne, Indoramine, Abanoquil, Corynanthine, Ajmalicine, Quetiapine, Dapiprazone, Amoxapine, Risperidone, Idazoxan, Bunazosin, Doxepin, Atiprosin, Piperoxan, Fenmetozole, Denopamine, Dobutamine, Dopexamine, Epinephrine, Isoprenaline, Isoproterenol, Prenalterol, Xamotero, Arformoterol, Buphenine, Clenbuterol, Dopexamine, Epinephrine, Fenoterol, Formoterol, Isoetarine, Levosalbutamol, levalbuterol, Orciprenaline, metaproterenol, Pirbuterol, Procaterol, Ritodrine, Salbutamol, albuterol, Salmeterol, Terbutaline, Arbutamine, Befunolol, Bromoacetylalprenololmenthane, Broxaterol, Cimaterol, Cirazoline, Etilefrine, Hexoprenaline, Higenamine, Isoxsuprine, Mabuterol, Methoxyphenamine, Oxyfedrine, Ractopamine, Reproterol, Rimiterol, Tretoquinol, Tulobuterol, Zilpaterol, Zinterol, Propranolol, Bucindolol, Carteolol, Carvedilol, Labetalol, Nadolol, Oxprenolol, Penbutolol, Pindolol, Sotalol, Timolol, Acebutolol, Atenolol, Betaxolol, Bisoprolol, Celiprolol, Metoprolol, Nebivolol, Esmolol, Butaxamine, ICI-118,551, SR 59230A, 4-NEMD, 7-Me-marsanidine, Agmatine, Apraclonidine, Brimonidine, Cannabigerol, Clonidine, Detomidine, Dexmedetomidine, Fadolmidine, Guanabenz, Guanfacine, Lofexidine, Marsanidine, Medetomidine, Methamphetamine, Mivazerol, Rilmenidine, Romifidine, Talipexole, Tiamenidine, Tizanidine, Tolonidine, Xylazine, Xylometazoline, L-748,328, L-748,337 and SR 59,230A, Amibegron (SR-58611A), CL-316,243, L-742,791, L-796,568, LY-368,842, Mirabegron (YM-178), Ro40-2148, Solabegron (GW-427,353), and Vibegron (MK-4618); hexamethonium, pentolinium, mecamylamine, trimetaphan, tubocurarine, pempidine, benzohexonium, chlorisondamine, pentamine, and tetra ethylammonium chloride.

Similarly to the above adrenergic blocking agents, muscarinic cholinergic receptor blocking agents, or any combination of muscarinic or nicotinic cholinergic receptor blocking agents, (denominated cholinergic blocking agents from here on) including, but not limited to, the compounds listed in the following list can be used for this purpose:

Atropine, scopolamine, homatropine, tropicamide, benzproine, biperiden, trihexyphenidyl, oxybutynin, tolterodine, solifenacin, dicyclomine, darifenacin, glycopyrrolate, ipratropium, and tiotropium.

In addition, the above adrenergic blocking agents and cholinergic blocking agents can be used in combination with any anti-seizure medicine including, but not limited to, the following examples:

Phenytoin, carbamazepine, clobazam, divalproex, eslicarbazepine, oxcarbazepine, lacosamide, ethosuximide, gabapentin, methsuxamide, perampanel, phenobarbital, pregabalin, rufinamide, tiagabine, vigabatrin, levetiracetam, clonazepam, clorazepate, ezogabine, felbamate, lamotrigine, lorazepam, primidone, topiramate, valproic acid, and zonisamide.

For instance, a pill formulation that contains both phenytoin and phentolamine can be prescribed in lieu of phenytoin alone to patients whose risk of death from seizures has been found to be elevated by the above methods and test kits, especially during times when patients are suffering from uncontrolled seizures as the increased frequency of seizures further increases the risk of death from seizures. Another example to be used in the same fashion is a formulation that combines levetiracetam and oxybutynin, which can be used in lieu of levetiracetam alone.

A scientific study titled “post-convulsive leucocytosis as a biomarker for peri-ictal respiratory collapse” is included in the pages that follow (immediately below). It shows that, in a population of patients seen at an emergency department for the care of epileptic convulsions, those who demonstrated respiratory signs that demanded emergent life-saving endotracheal intubation demonstrated the highest levels of white blood cells, platelets, and lactic acid in the peripheral blood. They also demonstrated the highest pulse rates. The study offers evidence to support the basis for this invention, namely that the higher these blood markers are, the higher the chance of experiencing respiratory failure, which could result in sudden death after a seizure.

Abstract: a fraction of generalized convulsive seizures (GCS) is accompanied by a transient post-convulsive leucocytosis (PoCL) whose clinical significance remains unclear. In addition, an apparently independent subset of GCS exhibits signs and symptoms of peri-convulsive respiratory compromise (PeCRC), which in the worst cases can lead to death. In this study we tested the hypothesis that PoCL is physiologically linked to PeCRC. To this end, we performed a retrospective chart review of 152 patients who presented to a large tertiary care facility with GCS between Jan. 1, 2017 and Aug. 23, 2018, in order to investigate the relationship between PoCL and PeCRC. Our data not only establish an association between these two GCS epiphenomena, but suggest that PoCL is a continuum whose magnitude is directly proportional to the degree of PeCRC. Moreover, by uncovering a faithful correlation between the post-convulsive white blood cell (WBC) count and the peri-convulsive heart rate, this study suggests that the mechanism driving PeCRC stems from a surge in cardiac output. Importantly, the area under the curve obtained from a receiving operating characteristic analysis (0.81) indicates that the post-convulsive WBC count is closely associated with respiratory symptoms which commonly instigate a decision to perform emergent endotracheal intubation. Thus, in the aggregate, our data suggest that PoCL is an effective biomarker for a peri-convulsive pathological process capable of causing respiratory collapse, and potentially, sudden unexpected death.

Introduction: sporadically, epileptic seizures generate a transient and noninfectious elevation of the white blood cell (WBC) count of unclear clinical significance. This seizure epiphenomenon, herein referred to as post-convulsive leucocytosis (PoCL), occurs in 7% of self-limiting complex partial seizures but in up to 63% of generalized convulsive seizures (GCS) associated with convulsive status epilepticus (CSE), suggesting that the pathophysiological process driving it, is associated with both seizure intensity and clinical outcome. As peripheral catecholamine injections induce a similar leucocytosis, some authors have suggested that PoCL is driven by systemic catecholamines. However, while this notion is congruent with prior demonstrations of increased blood catecholamine levels following GCS, the relationship between post-convulsive catecholamines and PoCL has to our knowledge never been investigated. Thus, the pathophysiological forces driving PoCL have yet to be described.

Another sporadic seizure epiphenomenon associated with convulsive intensity is peri-ictal respiratory compromise (PeCRC). Like PoCL, PeCRC is observed across a wide range of epileptic semiologies, but arises most commonly, and most severely, in association with GCS. However, unlike PoCL, which is thought to bear little or no consequence in the diagnosis or management of GCS, PeCRC can be severe, require emergent endotracheal intubation and, in the worst cases, it can cause death. In fact, sudden unexpected death in epilepsy (SUDEP), an enigmatic condition that takes the lives of thousands of otherwise healthy epilepsy patients every year, is most frequently associated with overt peri-ictal respiratory abnormalities, and upwards of 70% of its victims demonstrate pulmonary edema and/or pulmonary hemorrhage at autopsy.

In this study, we tested the hypothesis that PoCL and the signs and symptoms of PeCRC share a common pathophysiological origin. To this end, we performed a retrospective chart review to quantify the frequency with which PoCL and PeCRC coexist.

Our results not only reveal a strong correlation between these two seemingly unrelated seizure epiphenomena, but suggest, for the first time, that PoCL could serve as a biomarker for life-threatening peri-ictal respiratory failure.

Methods

Patient Selection and Determination of PeCRC and PoCL

This retrospective chart review was performed at the Forsyth Medical Center, a large tertiary care hospital in Winston Salem, N.C., with approval from its institutional review board. The study included patients admitted between Jan. 1, 2017 and Aug. 23, 2018, for the treatment of convulsive seizures with or without status epilepticus. These diagnoses were chosen due to their previously demonstrated association with PoCL. We used ICD-10 codes R56.9 (seizures, 80.4%) and G40.901 (status epilepticus, 19.6%) to generate a patient source list (n=446) from which we pulled electronic medical records (EMRs) demonstrating unequivocal evidence of at least one witnessed GCS occurring immediately prior to arrival at the emergency department (ED), or inside the ED. For patients admitted for GCS more than once during the study period, only the earliest EMR was included in the patient source list. EMRs reflecting erroneous diagnoses (n=73), non-convulsive (n=101) or non-epileptic (n=13) semiologies, those lacking a postictal complete blood count drawn within the first 12 h of admission (CBC1; n=67), exhibiting evidence of recent or active infections (n=26), or the possibility of abnormal immunological function due to disease (e.g., HIV+ status) or due to active treatment with chemotherapy, steroids, or other immunosuppressant agents (n=14), were excluded from the study.

The remaining patients (n=152) were assigned to one of two groups depending on their peri-ictal respiratory status. The PeCRC+ group included patients whose EMRs contained unequivocal written descriptions of ictal or postictal respiratory compromise, or evidence of endotracheal intubation. The latter was defined as definitive placement of a tracheal airway followed by a period of mechanical ventilation. The PeCRC− group included the remaining patients. Written descriptions of respiratory compromise—as provided by witnesses—included expressions such as “labored breathing,” “cyanosis,” “stopped breathing”, “could not breathe,” “ . . . was gasping for air,” and “ . . . lips turned blue,” among others. Patients were also classified according to the initial postictal white blood cell count (WBC1) obtained from CBC1. As previous publications on this subject defined PoCL as an acute and transient elevation of the WBC count above its normal range in the context of an epileptic convulsion,^{1,2} patient's whose WBC1 counts fell above the upper limit of the normal WBC range at our institution (>11,000 cells/mm³) were included in the PoCL+ group, while patients whose WBC counts fell below this limit were included in the PoCL− group. Initial postictal hemoglobin, ED triage vital signs, and initial results of chest X rays (CXR) obtained within 36 h of ED triage were also recorded (see Methods).

Confirmatory PoCL Patient Selection

While our patient selection criteria was designed to exclude spuriously elevated WBC1 counts, we also tested the validity of our initial results through a confirmatory statistical analysis of a subgroup of patients whose WBC1 elevations could be demonstrated to be transient against a follow-up WBC count performed within 36 hours of WBC1 (WBC2). This time limit was chosen based on our experience with PoCL, as little has been written about the kinetics of PoCL responses. Thus, patients lacking a follow-up CBC obtained within this 36 h time limit (CBC2) and patients in the PoCL+ group whose WBC2>WBC1 were excluded from this part of the analysis. Patients in the PoCL− group whose WBC2>WBC1 were not excluded.

Assessment of Relative PoCL (RPoCL) Responses.

To ascertain whether RPoCL responses occur within the normal range of the WBC count we computed an index arbitrarily named relative leucocytosis index (RLI) calculated by the following equation: (WBC1-WBC2)/(WBC1). We assumed two possible outcomes for the relationship between WBC1 and WBC2: 1) WBC1<WBC2 and 2) WBC1>WBC2). The third possible outcome, WBC1=WBC2, was not considered as none of our patients demonstrated such a result. We hypothesized that following GCS, the outcome WBC1>WBC2 would occur much more frequently than expected by chance. Thus, WBC1 counts ≥11,000 cells/mm which demonstrated RLIs>0 were included in the PoCL+ group, while WBC1 counts<11,000 cells/mm³ demonstrating RLIs>0 were interpreted as being relative PoCL (RPoCL) responses. All RLIs<0 were included in the PoCL− group.

Assessment of the Effect of Hemodilution on Postictal WBC Counts

As hemodilution from continuous infusion of intravenous fluids in the hospital could have decreased the WBC2 concentration and mimic an outcome in which WBC1>WBC2, the need arose to ascertain whether the decrease in WBC count observed between CBC1 and CBC2 in PoCL+ and RPoCL responses exceeded that expected by hemodilution. To accomplish this, we compared the normalized change in WBC count observed between these two blood samples with the corresponding normalized change in hematocrit (HCT). For normalization, the smallest change in WBC count and hematocrit between CBC1 and CBC2 for each data set was defined as 0%, and the largest change as 100%.

Receiving Operating Characteristic (ROC) Curves

ROC curves were generated to summarize the overall performance of WBC1 and heart rate as possible predictors of endotracheal intubation, which for the purposes of this study was interpreted as evidence of respiratory collapse. An area under the curve (AUC) value of 0.5 suggests a predictive performance not different from chance. An AUC value of 1.0 suggests a predictive performance with 100% sensitivity and specificity.

Quantification of Chest X Ray (CXR) Abnormalities

To investigate the presence of acute CXR abnormalities in our GCS cohort, we reviewed available reports of postictal CXRs acquired no later than 36 h after admission (n=107). CXRs demonstrating chronic pulmonary pathology, (e.g., pulmonary nodules or masses) or cardiac abnormalities (e.g., cardiomegaly), were excluded from this part of the analysis. Patients were assigned to a CXR− (normal) or to a CXR+(i.e., abnormal) group, depending on results dictated by radiologists in the context of routine patient care. C×R images were not reviewed as part of this study. The CXR− group (n=64) included only readings described by terminology which denoted normal findings. Such readings included terms such as “normal”, “nothing acute”, and “no acute cardiopulmonary disease.” All other CXR readings (n=43) were included in the CXR+ group.

Data Analysis

Continuous variables were reported as mean±standard deviation (SD) and, when necessary, compared using paired or unpaired parametric and non-parametric t-tests, as appropriate. Categorical variables were reported as whole numbers and compared via Fishers exact test. Relationships between relevant continuous variables were assessed using linear regression. Normalized HCT changes were compared with their corresponding WBC count changes via two-tailed Wilcoxon matched pairs sign-ranked test. The frequency of RLIs>0 was tested using a binomial test whose null hypothesis assumed equal outcomes for the relationship between WBC1 and WBC2 (see above). Outliers were not excluded. Statistical significance was defined by P values<0.05. SAS version 9.0 (SAS Institute, Cary, N.C.), and Prism version 8.0 were used for data analysis and graph design.

Results

Demographic Characteristics of PeCRC+ and PoCL+ Patients

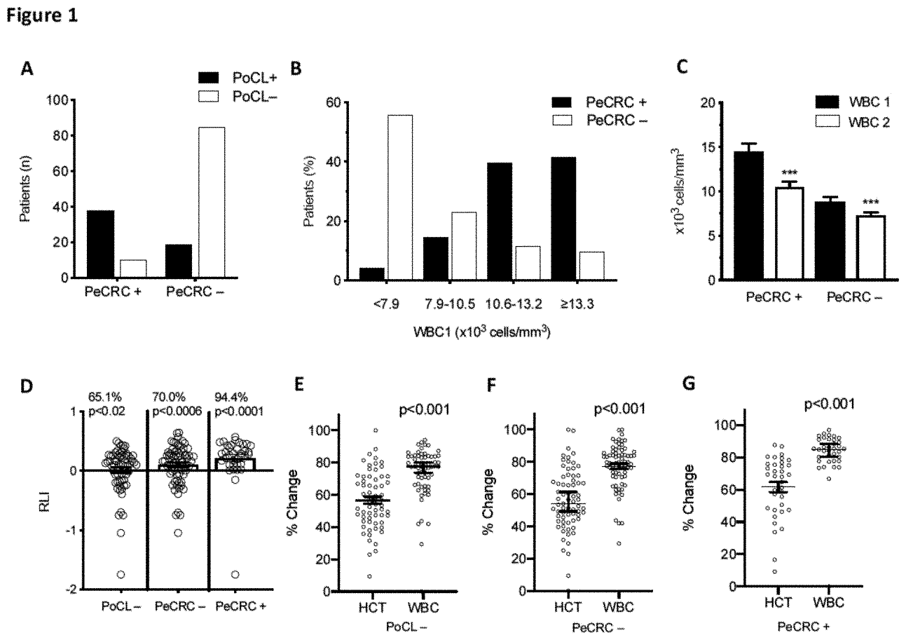

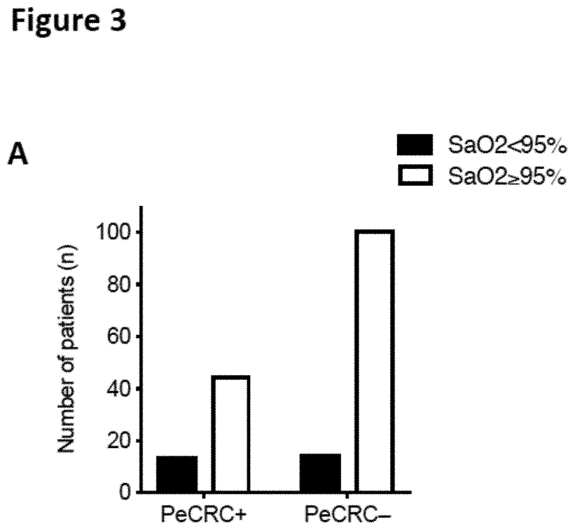

Demographic characteristics, average times from triage to blood collection, from triage to CXR acquisition, and other continuous variables for the entire cohort, were statistically similar across all subgroups (Table 1). All patients in the PeCRC+ group contained clear descriptions of respiratory compromise in their EMR's. A larger fraction of PeCRC+ patients demonstrated oxygen saturations<95%, than PeCRC− patients, (OR=2.1; 95% CI=0.94-4.6; p>0.09; Fishers exact test; FIG. 3; Table 2). We attribute the lack of statistical significance to the routine use of high O₂ concentrations in PeCRC+ patients, which brings their O₂ saturations closer to the normal baseline. However, documentation of O₂ concentrations administered to patients before arrival, or at triage, were not available for the majority of patients and therefore were not included as part of this investigation. Forty-eight (31.6%) patients were PeCRC+, and twenty-one of them (43.8%) required endotracheal intubation (PeCRC+ INT+; Table 1). Relevant aspects of the clinical history of each PeCRC+ INT+ patient are described in Table 4. Fifty-seven patients (37.5%) were PoCL+. PoCL coexisted with PeCRC in 38/48 PeCRC+ patients (79.2%), but only in 19/104 (18.2%) PeCRC− patients (FIG. 1A; Table 1), suggesting that PeCRC and PoCL share a common pathophysiological origin. In addition, the WBC1 count of PeCRC+ and PeCRC− patients exhibited opposite distributions across the cohort's WBC1 count range (3.3-31.2; Table 4), with the majority of PeCRC+ patients falling in its two highest quintiles, and the majority of PeCRC− patients falling in its three lowest quintiles (FIG. 1B; Table 1). Statistics and conclusions drawn from the original cohort were validated by the confirmatory group analysis, except for the elevated temperature found in intubated patients (Table 1), which could not be confirmed (Table 6).

PoCL is Maximal in PeCRC+ Patients

The WBC1 count of PeCRC+ patients averaged 14.4±5.5×10³ cells/mm³ (FIG. 1C) and, as expected, demonstrated a rapid (17.7±9.7 hours) return to the normal WBC count range (FIG. 1C; Table 6). By contrast, in PeCRC− patients, the WBC1 count remained within the normal WBC count range, averaging 8.8±4.3×10³ cells/mm³. Yet these patients also experienced a small but significant drop in their WBC counts within a similar time frame (FIG. 1C; 18.7±9.4 hours; Table 6).

PeCRC+ and PeCRC-Patients Experience PoCL and RPoCL Responses

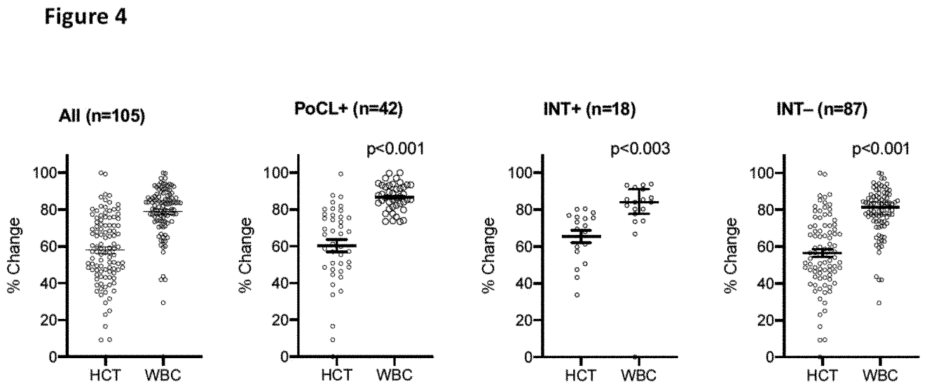

The majority of patients in this study demonstrated RLIs>0, including patients in the PoCL− group (FIG. 1D). This result indicates that GCS induce different degrees of post-convulsive leucocytosis, some of which occur within the normal WBC count range (i.e., RPoCL), and some of which occur outside of this range (i.e., PoCL). Notably, a striking majority of PeCRC+ patients exhibited RLI>0 (FIG. 1D), further suggesting a physiological link between postictal leucocytosis and PeCRC. These results are likely to reflect true leucocytosis responses and are unlikely to stem from dilutional causes, as post-convulsive changes in WBC count in all groups were statistically greater than their concomitant hematocrit changes (FIG. 1E-G; FIG. 4).

PoCL+ and PeCRC+ Patients Exhibit Increased Heart Rates and CXR Abnormalities

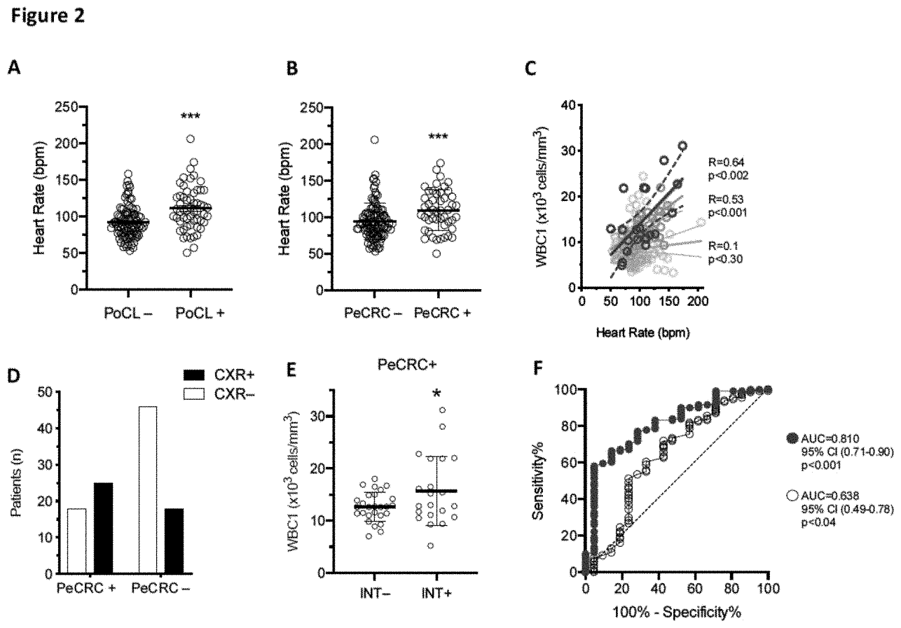

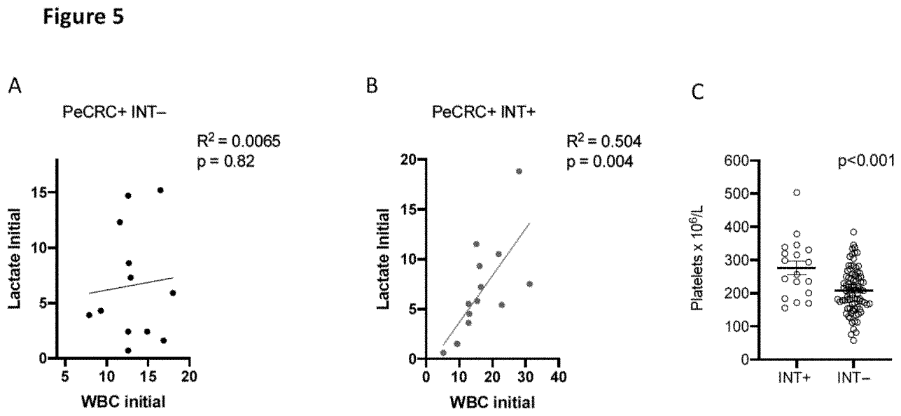

Both PoCL+ and PeCRC+ patients demonstrated increased heart rates (FIG. 2A-B; Tables 1 and 6). In addition, a strong positive correlation between WBC1 count and heart rate was seen in PeCRC+ patients (FIG. 2C), which was most significant among INT+ patients (FIG. 2C). These results demonstrate that PoCL mirrors the severity of respiratory symptoms in GCS patients, a notion that is exemplified by our finding that all PeCRC+ patients whose WBC1 exceeded 18,000 cells/mm³ required endotracheal intubation (FIG. 2E; Table 3). Of all the patients whose hospital care included a CXR obtained within 36 h of admission (n=107; Table 7) PeCRC+ patients demonstrated the highest frequency of acute, though non-specific, CXR abnormalities (FIG. 2D; Tables 1 and 6). PeCRC induced a concurrent increase in heart rate and lactic acid (FIG. 5). This correlation is not observed in GCS who demonstrate PeCRC but who do not require emergent endotracheal intubation (FIG. 5). Finally, GCS patients who require emergent endotracheal intubation also demonstrated higher post-convulsive platelet levels than patients who do not required emergent endotracheal intubation (FIG. 5; Tables 1 and 6). These results suggest that heart, lactic acid, and platelets could play an important role in the identification of patients at risk of peri-convulsive respiratory collapse, and thus, of patients at risk of SUDEP.

The WBC1 Count Heralds the Need for Endotracheal Intubation

We then evaluated the performance of both WBC1 and heart rate as predictors of endotracheal intubation (i.e., respiratory collapse) via ROC. The AUC obtained from these tests reveals that the WBC1 count is indeed a strong predictor of such an outcome (FIG. 2F). Specific cutoff values and their corresponding sensitivities and specificities for both WBC1 and heart rate are shown in Tables 8 and 9, respectively.

PeCRC+ Patients Demonstrate Frequent Acute CXR Abnormalities

Sixty-four CXRs were normal (CXR−), and 43 were abnormal (CXR+). The latter contained a total of fifty-eight abnormalities, including 5 opacities, 13 infiltrates, 21 atelectasis, 6 pulmonary edema, 6 vascular congestion, 5 pleural effusions, and 2 increased interstitial markings. CXRs demonstrated one abnormality in 27 patients, two abnormalities in 14 patients and three in 1 patient. Findings were bilateral in 29 patients, and unilateral in 14. Both PeCRC+ and PoCL+ patients demonstrated a significantly higher frequency of CXR abnormalities than their respective PeCRC and PoCL− counterparts (Table 1; also see Table 6). However, PeCRC− PoCL+ patients did not demonstrate this effect (data not shown), indicating that postconvulsive CXR abnormalities are specifically associated with PeCRC, and not with PoCL.

### DISCUSSION

For decades, PoCL has been perceived as a mere seizure epiphenomenon devoid of clear clinical significance. In this article, for the first time, we present data suggesting that this seemingly random occurrence represents the footprint of a pathological process capable of causing respiratory decompensation. Specifically, we show that a majority of the GCS that cause subjective and/or objective signs of respiratory compromise also exhibit PoCL (FIG. 1A-B). In this patient population, the risk of respiratory decompensation increases proportionally with the WBC count, reaching a threshold around 18,000 cells/mm³ beyond which all patients required endotracheal intubation (FIG. 2E; Table 3). Moreover, the assessment of WBC1 count's performance as a predictor of endotracheal intubation (FIG. 2F) yielded an AUC of 0.81, suggesting that PoCL is a suitable biomarker for this pathological process. While prior publications had noted a strong correlation between CSE and PoCL, this is the first time that data are presented to support a specific link between peri-ictal respiratory symptoms and PoCL.

Our data also invite a reassessment of the widespread notion that PoCL is limited to abnormal WBC count elevations. This can be deduced from our findings that a significant majority of patients whose post-ictal WBC counts fall within the normal range, also demonstrate RLIs>0 (FIG. 1D). Thus, while some GCS induce florid PoCL+ responses, most of them induce moderate RPoCL responses that remain within the normal reference range. If these milder responses are taken into consideration 65.1% (68/105) of PoCL-patients, 70% (48/69) of PeCRC− patients, and 94% (34/36) PeCRC+ patients, demonstrate some degree of postictal leucocytosis (FIG. 1D). As these results do not appear to be related to dilutional factors (FIG. 1 E-G; FIG. 4), they further support the notion that the severity of respiratory abnormalities associated with GCS mirror the magnitude of their corresponding WBC1 counts (FIG. 1A-C; FIG. 2E). Therefore, clinically significant PeCRC is much more likely to coexist with WBC1 counts that exceed the normal reference range than with WBC1 counts that remain within the normal reference range (FIG. 1A-C; FIG. 2E). This notion is consistent with the high frequency of PoCL associated with convulsive status epilepticus (CSE), as this clinical entity often induces severe respiratory abnormalities that require emergent endotracheal intubation.¹⁷ By the same token, this notion is consistent with the infrequent role of endotracheal intubation in the management of self-limiting non-convulsive seizures.

While some emergent peri-ictal intubations are performed to protect patients' airways, or in anticipation of respiratory suppression from the use of aggressive antiepileptic medication doses, many such intubations are performed to address respiratory abnormalities of unclear etiology. In our confirmatory analysis cohort, only two of eighteen patients were intubated for airway protection, and of the remaining sixteen, five of the six patients with the highest postictal WBC1 counts (range 21.9-31.2 cells/mm³) received 2 mg of lorazepam or less prior to intubation (Table 3). As this low benzodiazepine dose is unlikely to have suppressed their respiratory drives, the underlying etiology for these patients' severe respiratory symptoms, which ranged from respiratory distress to agonal breathing (Table 3), remains unexplained. We hypothesize that these severe respiratory symptoms stem from the same pathophysiological process that causes PoCL.

The elevated heart rate found in both PeCRC+ and PoCL+ groups (FIG. 2A-B) suggests that the pathological process propelling these periconvulsive phenomena is sparked by increased cardiac output. This is particularly likely in the setting of a high ictal catecholaminergic state,⁶ as a combination of pulmonary vasoconstriction with the increased pulmonary vascular load expected to accompany a surge in cardiac output, could precipitate transvascular fluid filtration and cause subtle but clinically significant pulmonary edema. Not only is this notion consistent with the strong correlation between heart rate and WBC1 count in PeCRC+ INT+ patients (FIG. 2C), but also with previous reports that prolonged GCS induce postictal CXR abnormalities akin to pulmonary edema. In addition, this mechanism could explain the high frequency of non-specific CXR abnormalities found in both PeCRC+(FIG. 2D), and in PeCRC+ INT+ patients in our own study (Tables 1, 3, 6), although the wide range of abnormalities reported in these patients, and the poor sensitivity and specificity of this diagnostic tool, prevent us from speculating further about the meaning of these results.

An increase in cardiac output, and in pulmonary blood flow, also provide a likely mechanism for post-convulsive leucocytosis. It has been known for some time that the pulmonary vasculature contains the body's largest pool of marginated leukocytes, and that increased pulmonary blood flow induces detectable elevations of the peripheral WBC count. Even minor increases in cardiac output arising from passive exhalation, and from voluntary breathing movements like the Muller maneuver (i.e., a deep inspiration against a closed glottis), have been associated with peripheral WBC count elevations. Thus, while in theory alternative leukocyte sources such as the spleen could help generate PoCL responses, a sudden increase in cardiac output precipitated by a peri-convulsive release of systemic catecholamines is a likely catalyst for both PoCL and PeCRC.

While it is not possible to determine how the PeCRC+ patients in our cohort might have fared in the absence of emergent medical care, it is reasonable to suspect that without endotracheal intubation some of these patients might have perished in the peri-ictal period. This notion is supported by a recent study which demonstrated a link between delayed peri-ictal intubations of CSE patients and increased mortality. In our cohort, 62% (13/21) of the patients who required endotracheal intubation received a diagnosis of CSE, and of these, 77% (10/13) demonstrated a PoCL+ response (Table 3).

In conclusion, this is the first demonstration that PoCL mirrors peri-convulsive respiratory symptom severity, a novel finding which implicates the post-convulsive WBC count as a possible biomarker for an ictal pathophysiological process that could result in respiratory collapse, and thus, in SUDEP.

FIG. 1 shows postictal WBC count dynamics in the presence and absence of periconvulsive respiratory compromise (PeCRC). A. Postconvulsive leucocytosis (PoCL) occurred most frequently in patients who experienced PeCRC (PeCRC+), and only rarely in patients who did not experience PeCRC (OR=17.0; 95% CI 7.2-40.9; p<0.0001). B. PeCRC+ patients most frequently demonstrated initial white blood cell (WBC1) counts in the upper two quintiles, while PeCRC− patients most frequently demonstrated WBC1 counts in the two lowest quintiles of the WBC count range (3,300-31,200 cells/mm³; p<0.001; Chi square test for trend). C. WBC1 elevations experienced a significant decrease within the first 24 h. Such elevations occurred beyond the normal WBC range for the PeCRC+ group of patients, but within the normal range for the PeCRC− group. Error bars represent the mean s.e.m. ***p<0.001. D. The relative leucocytosis index (RLI) was positive in the groups shown at much higher rates than expected by chance, including the PoCL− group. Notably, the highest frequency of positive RLIs was seen in the PeCRC+ group. The percentages written above the top panels represent the observed frequency with which the RLI was positive for each group; the p values show the significance of a binomial test comparing each observed frequency with an expected frequency of 50%. E-G. Comparison of normalized WBC count and hematocrit changes from initial complete blood count performed within 12 hours of arrival at the emergency department (CBC1) to a blood count performed within 36 hours of CBC1 (CBC2). The p values above each graph show the significance of within group paired comparisons using a two-tailed Wilcoxon matched pairs sign-ranked test. Error bars show the mean±s.e.m. Graphs for the entire cohort, for PoCL− patients, for patients who did not require endotracheal intubation (INT−) and for patients who required endotracheal intubation (INT+) can be found in FIG. 4. HCT=hematocrit; WBC=white blood cell count.

FIG. 2 shows the relationship between PeCRC, WBC1 count, heart rate, and endotracheal intubation. A-B. PeCRC+ and PoCL+ patients exhibited increased heart rates compared with their PeCRC- and PoCL− counterparts. Error bars show mean±s.e.m. C. A correlation between heart rate and WBC1 is evident in PeCRC+ patients (n=48; pink circles) and is particularly strong among PeCRC+ INT+ patients (n=21; blue circles). Note that this correlation is no longer present when tested in the entire patient cohort (n=152; orange circles). P values represent significance of goodness of fit data obtained via linear regression. D. PeCRC+ patients exhibit acute CXR abnormalities more frequently than PeCRC− patients (p<0.003; 95% CI=1.5-8.0; Fishers exact test; also see Table 1). E. The mean WBC1 count of PeCRC+ patients who required intubation (INT+) significantly exceeded that 0f PeCRC+ patients who did not require intubation (INT−). Note that all PeCRC+ patients whose WBC count exceeded 18,000 cells/mm³ required intubation. *p<0.05; Error bars show mean s.e.m. F. area under the curve (AUC) obtained from a receiving operator characteristic (ROC) analysis of WBC1 and heart rate performance as biomarkers for endotracheal intubation (i.e., respiratory collapse). The performance of WBC1 is particularly strong, while, while still significant, that of heart rate is modest. Specific ROC cutoff values for WBC1 and heart rate are shown in the Tables 8 and 9, respectively.

FIG. 3 shows triage pulse oximetry in PeCRC+ and PeCRC− patients. Fraction of patients in each group that demonstrated O2 saturations <95%. While a trend is seen towards increased desaturations in PeCRC+ patients, this finding was not statistically significant (OR=2.1; 95% CI=0.94-4.6; p>0.09; Fishers exact test). PeCRC=peri-ictal respiratory compromise. SaO2=oxygen saturation measured by pulse oximetry at ED triage.

FIG. 4 shows normalized postconvulsive changes in WBC count and hematocrit. Comparison of percent normalized WBC count and hematocrit changes from CBC1 to CBC2. The p values above each graph show the significance of paired comparisons using a two-tailed Wilcoxon matched pairs sign-ranked test. Error bars show the mean±s.e.m. Graphs for PoCL−, PeCRC− and PeCRC+ groups can be found in the main text (FIG. 1E-G, respectively). HCT=hematocrit; WBC=white blood cell count.

Table Descriptions:

Tables 2, 4, and 5 are embedded in the text below but tables 1, 3, 6, 8 and 9 are located at the end of this text.

Table 1 shows continuous and categorical data and statistics summary for the entire GCS patient cohort (n=152). Cells in the first column and row, describe the type of data presented. The column labelled TOTAL shows data for the entire cohort unless a subgroup (e.g., PeCRC+) is specified. When appropriate categorical data from these groups appear in bold inside discrete 2×2 or 2×4 contingency sub-tables matched with subgroups described in corresponding column and row headings. These contingency sub-tables were analyzed via Fishers exact test, or Chi square test for trend, respectively. P values are located to the immediate right of each sub-tables and are bolded when statistically significant. Continuous variables shown by corresponding rows and columns were compared via parametric or non-parametric statistical tests, as appropriate. Abbreviations used are defined in alphabetical order immediately below the table.

Table 2 shows triage pulse oximetry (TPO) in PeCRC+ and PeCRC− patients. Available pulse oximetry values were analyzed using a cutoff value of 95%. The data obtained were compared between patients whose EMIR demonstrated descriptions of peri-convulsive respiratory compromise (PeCRC+) vs. those who did not (PeCRC−). A graph of these data is shown in FIG. 3.

TABLE 2
Triage pulse oximetry in PeCRC+ and PeCRC− patients (n = 152)

| | Original | With | |
|---|---|---|---|
| Group | Analysis (N) | TPO | TPO <95% |
| PeCRC+ | 48 | 45 | 14 |
| PeCRC− | 104 | 101 | 15 |

Table 3 shows clinical characteristics of patients who required endotracheal intubation. Each row shows the clinical characteristics of one GCS patient who experienced peri-convulsive respiratory compromise (i.e., PeCRC+) and required endotracheal intubation. Patients highlighted in orange were excluded from the confirmatory analysis as described in Table 5. Antiepileptic medications listed include all doses given before endotracheal intubation. The CXR column lists the main diagnosis described in reports dictated by reading radiologists (see Methods and Results). A plus sign under the EEG column indicates that either seizures or sharp waves were found on the patient's EEG. HR the heart rate recorded at the time of patient arrival at ED triage. Possible seizure etiology was estimated from the patient's clinical history. Abbreviations are defined in alphabetical order immediately below the table.

Table 4 shows WBC1 quintile distribution (range 3,300-31,200 cell/mm³). Quintile cutoff values, shown in the right hand column, were obtained from Microsoft Excel function “=percentile.inc(array,k)”, in which k is the percentile shown in the left hand column.

TABLE 4
WBC1 quintile distribution (range 3,300-31,200 cell/mm³)

| | WBC1 |
|---|---|
| Quintile | (cells/mm³) |
| 0.8 | 13,200 |
| 0.6 | 10,500 |
| 0.4 | 7,900 |
| 0.2 | 6,200 |

Table 5 shows confirmatory analysis patients by group (n=105). Numbers of patients within each subgroup included in the confirmatory analysis. The column with CBC2 refers to the number of patients whose charts contained a follow up CBC obtained within 36 h of CBC1. The column under “PoCL+ WBC1<WBC2” refers to the number of patients in each group, denoted by the corresponding row heading on the left side, who were excluded from this analysis because they demonstrated a post-convulsive leucocytosis based on the WBC1 count, which failed to decrease by the time it was remeasured on CBC2.

TABLE 5
Confirmatory analysis patients by group (n = 105)

| | Original | | PoCL+ | |
|---|---|---|---|---|
| Group | Analysis (N) | With CBC2 | WBC1 < WBC2 | Total |
| PeCRC+ | 48 | 38 | 2 | 36 |
| PeCRC− | 104 | 70 | 1 | 69 |
| PoCL+ | 57 | 45 | 3 | 42 |
| PoCL− | 95 | 63 | NA | 63 |
| INT+ | 21 | 20 | 2 | 18 |
| INT− | 131 | 87 | 0 | 87 |

Table 6 shows continuous and categorical data and statistics summary for the confirmatory analysis patient cohort (n=105). Cells in the first column and row, describe the type of data presented. The column labelled TOTAL shows data for the entire cohort unless a subgroup (e.g., PeCRC+) is specified. When appropriate categorical data from these groups appear in bold inside discrete 2×2 or 2×4 contingency sub-tables matched with subgroups described in corresponding column and row headings. These contingency sub-tables were analyzed via Fishers exact test, or Chi square test for trend, respectively. P values are located to the immediate right of each sub-tables and are bolded when statistically significant. Continuous variables shown by corresponding rows and columns were compared via parametric or non-parametric statistical tests, as appropriate.

Table 7 shows available of CXRs by group. Number of patients whose charts contained CXR reports of films obtained within the first 36 h of the patient's admission. Data presented relates to categorical comparisons shown in FIG. 2C, and in some of relevant contingency tables in Tables 1 and 6.

TABLE 7
Available CXRs by group

| | Original | | | Original | Confirmatory |
|---|---|---|---|---|---|
| | Analysis | Reanalysis | | Analysis | Analysis |
| Group | (N) | (N) | CXRs <36 h | with CXRs | with CXRs |
| PeCRC+ | 48 | 36 | 43 | 43 | 35 |
| PeCRC− | 104 | 69 | 64 | 64 | 38 |
| PoCL+ | 57 | 42 | 46 | 46 | 38 |
| PoCL− | 95 | 63 | 61 | 61 | 40 |
| INT+ | 21 | 18 | 21 | 21 | 18 |
| INT− | 131 | 87 | 87 | 87 | 60 |

Table 8 shows ROC table for the performance of WBC1 as a predictor of intubation, and Table 9 shows ROC table for the performance of initial triage heart rate as a predictor of intubation.

It will be apparent to those skilled in the art that various modifications and variations can be made in the present invention without departing from the spirit or scope of the invention. Thus, it is intended that the present invention cover the modifications and variations of this invention.

TABLE 1
Continuous and categorical data and statistics summary for the entire GCS patient cohort (n = 152)

| | TOTAL | PeCRC+ vs PeCRC− | | P | PoCL+ vs PoCL− | | P |
|---|---|---|---|---|---|---|---|
| N (%) | 152 (100) | 48 (31.6) | 104 (68.4) | — | 57 (37.5) | 95 (62.5) | — |
| PIRC+ N (%) | 48 (31.6) | — | — | — | 38 | 10 | 0.0001 |
| PIRC− N (%) | 104 (68.4) | — | — | — | 19 | 85 | |
| WBC1; mean ± SD | 10.2 ± 5.0 | 14.0 ± 5.0 | 8.4 ± 3.9 | 0.0001 | 15.2 ± 4.4 | 7.2 ± 1.9 | 0.0001 |
| WBC1 <7.9; N (%) | 60 (39.5) | 2 | 58 | 0.001 | — | — | — |
| WBC1 7.9-10.5; N (%) | 31 (20.4) | 7 | 24 | | — | — | — |
| WBC1 10.6-13.2 N (%) | 31 (20.4) | 19 | 12 | | — | — | — |
| WBC1 13.3-31.2 N (%) | 30 (19.7) | 20 | 10 | | — | — | — |
| Hemoglobin; mean ± SD | 13.1 ± 1.9 | 13.1 ± 2.3 | 13.1 ± 1.6 | 0.74 | 13.2 ± 2.1 | 13.0 ± 1.7 | 0.60 |
| Platelets ×10³; mean ± SD | 233.4 ± 75.8 | 265.7 ± 77.6 | 203.9 ± 66.8 | 0.0001 | 261.7 ± 79.4 | 200.5 ± 63.6 | 0.0001 |
| Hours to WBC1 mean ± SD | 1.1 ± 1.4 | 1.2 ± 1.5 | 1.0 ± 1.4 | 0.51 | 1.2 ± 1.6 | 1.0 ± 1.3 | 0.75 |
| CXR+ | 43 (40.2) | — | — | — | 24 | 19 | 0.028 |
| CXR− | 64 (59.8) | — | — | — | 22 | 42 | |
| Hours to CXR; mean ± SD | 401.9 ± 20.1 | 309.4 ± 767. | 457.2 ± 1166 | 0.24 | 290.1 ± 744.7 | 481.5 ± 1188 | 0.16 |
| Age; years mean ± SD | 61.1 ± 18.2 | 60.5 ± 18.6 | 61.5 ± 18.1 | 0.75 | 60.1 ± 17.8 | 61.8 ± 18.5 | 0.58 |
| Male vs | 69 (45.4) | 22 | 47 | 0.99 | 30 | 39 | 0.18 |
| Female; N (%) | 83 (54.6) | 26 | 57 | | 27 | 56 | |
| SBP mean ± SD | 147.7 ± 35.7 | 147.1 ± 41.8 | 148 ± 32.8 | 0.68 | 152 ± 40.21 | 145 ± 30.7 | 0.25 |
| DSP mean ± SD | 82.2 ± 20.1 | 81.9 ± 19.8 | 82.3 ± 20.3 | 0.92 | 84.1 ± 22.1 | 81.0 ± 18.9 | 0.35 |
| Heart rate; mean ± SD | 99.0 ± 26.9 | 109.3 ± 28.0 | 94.3 ± 25.1 | 0.001 | 110.8 ± 30.2 | 91.6 ± 22.0 | 0.0001 |
| Temperature; mean ± SD | 98.5 ± 1.2 | 98.5 ± 28.2 | 98.4 ± 1.0 | 0.26 | 98.6 ± 1.2 | 98.4 ± 1.2 | 0.43 |

| | CXR+ vs CXR− | | P | INT+ vs INT− | | P |
|---|---|---|---|---|---|---|
| N (%) | 43 (40.2) | 64 (59.8) | — | 21 (13.8) | 131 (86.2) | — |
| PIRC+ N (%) | 25 | 18 | 0.003 | — | — | — |
| PIRC− N (%) | 18 | 46 | | — | — | — |
| WBC1; mean ± SD | 12.0 ± 5.2 | 10.1 ± 4.9 | 0.06 | 15.7 ± 6.6 | 9.3 ± 4.0 | 0.0001 |
| WBC1 <7.9; N (%) | 8 | 25 | 0.02 | 1 | 59 | 0.001 |
| WBC1 7.9-10.5; N (%) | 9 | 15 | | 3 | 28 | |
| WBC1 10.6-13.2 N (%) | 15 | 12 | | 7 | 24 | |
| WBC1 13.3-31.2 N (%) | 11 | 12 | | 10 | 20 | |
| Hemoglobin; mean ± SD | 12.7 ± 2.1 | 13.2 ± 1.8 | 0.15 | 13.4 ± 2.3 | 13.0 ± 1.8 | 0.40 |
| Platelets ×10³; mean ± SD | 2.17 ± 81.2 | 227.4 ± 78.2 | 0.53 | 272.0 ± 88.6 | 212.3 ± 74.2 | 0.001 |
| Hours to WBC1 mean ± SD | 0.9 ± 0.8 | 1.3 ± 1.5 | 0.20 | 1.1 ± 1.0 | 1.1 ± 1.5 | 0.001 |
| CXR+ | — | — | | 14 | 29 | 0.007 |
| CXR− | — | — | | 7 | 58 | |
| Hours to CXR; mean ± SD | 193.4 ± 300.1 | 160.9 ± 240.8 | 0.41 | 157.4 ± 168.62 | 454.5 ± 1124 | 0.001 |
| Age; years mean ± SD | 64.7 ± 16.8 | 63.3 ± 16.6 | 0.68 | 57.7 ± 13.9 | 61.7 ± 18.7 | 0.35 |
| Male vs | 18 | 33 | 0.43 | 8 | 61 | 0.49 |
| Female; N (%) | 25 | 31 | | 13 | 70 | |
| SBP mean ± SD | 140.3 ± 39.7 | 153.1 ± 35.4 | 0.08 | 152 ± 48.6 | 147 ± 33.4 | 0.72 |
| DSP mean ± SD | 77.6 ± 21.1 | 86.2 ± 21.1 | 0.04 | 88.1 ± 22.2 | 81.2 ± 19.7 | 0.15 |
| Heart rate; mean ± SD | 102.4 ± 27.7 | 99.7 ± 27.0 | 0.62 | 111.6 ± 33.6 | 97.0 ± 25.2 | 0.04 |
| Temperature; mean ± SD | 98.7 ± 1.8 | 98.4 ± 1.0 | 0.43 | 98.7 ± 2.1 | 98.4 ± 1.0 | 0.03 |

WBC1 = Initial postictal white blood cell count;
PeCRC+ = Periconvulsive respiratory compromise;
PeCRC− = Absence of periconvulsive respiratory compromise;
PIL+ = Postictal leukocytosis, defined as WBC1 count ≥11,000 cells/mm³;
PIL− = postictal WBC1 count <11.000 cells/mm³;
CXR+ = Abnormal chest X ray;
CXR− = Normal chest X ray;
INT+ = Intubated;
INT− = Not intubated;
Min. = minutes;
SBP = Systolic blood pressure;
DBP = Diastolic blood pressure

TABLE 6
Confirmatory analysis continuous and categorical data and statistics summary

| | TOTAL | PeCRC+ vs PeCRC− | | P | PoCL+ vs PoCL− | | P |
|---|---|---|---|---|---|---|---|
| N (%) | 105 (100) | 36 | 69 | — | 42 | 63 | |
| PIRC+ N (%) | 36 (33.9) | — | — | — | 29 | 7 | 0.001 |
| PIRC− N (%) | 69 (66.1) | — | — | — | 13 | 56 | |
| WBC1; mean ± SD | 10.2 ± 5.0 | 14.4 ± 5.5 | 8.8 ± 4.3 | 0.001 | 15.9 ± 4.9 | 7.3 ± 1.9 | 0.001 |
| Hours to WBC1; mean ± SD | 1.1 ± 1.6 | 1.2 ± 1.7 | 1.1 ± 1.6 | 0.61 | 1.3 ± 1.8 | 1.0 ± 1.5 | 0.51 |
| WBC2; mean ± SD | 8.4 ± 3.5 | 10.5 ± 3.6 | 7.3 ± 2.9 | 0.001 | 10.6 ± 3.7 | 6.9 ± 2.4 | 0.001 |
| Hours to WBC2; mean ± SD | 25.6 ± 28.6 | 17.7 ± 9.7 | 18.7 ± 9.4 | 0.58 | 20.2 ± 10.4 | 17.1 ± 8.6 | 0.11 |
| WBC1 <7.9; N (%) | 36 (34.3) | 1 | 36 | 0.001 | 0 | 36 | N/A |
| WBC1 7.9-10.5; N (%) | 24 (22.9) | 5 | 18 | | 0 | 24 | |
| WBC1 10.6-13.2; N (%) | 20 (19.0) | 15 | 6 | | 18 | 3 | |
| WBCl 13.3-31.2; N (%) | 25 (23.8) | 15 | 9 | | 24 | 0 | |
| Hemoglobin; mean ± SD | 13.1 ± 1.8 | 13.1 ± 2.1 | 13.1 ± 1.7 | 0.97 | 13.3 ± 1.9 | 13.0 ± 1.9 | 0.43 |
| Platelets ×10³; mean ± SD | 220.6 ± 78.9 | 262.0 ± 73.5 | 197.1 ± 65.2 | 0.001 | 259.1 ± 79.1 | 192.9 ± 61.7 | 0.001 |
| CXR+ | 35 (43.7) | — | — | — | 19 | 14 | 0.26 |
| CXR− | 45 (56.3) | — | — | | 19 | 26 | |
| Hours to CXR; mean ± SD | 6.7 ± 17.1 | 2.2 ± 2.4 | 10.0 ± 22.9 | 0.007 | 2.0 ± 2.4 | 10.7 ± 23.5 | 0.003 |
| Age; years mean ± SD | 61.1 ± 18.2 | 60.9 ± 17.8 | 60.5 ± 16.9 | 0.90 | 61.3 ± 16.9 | 60.1 ± 17.4 | 0.73 |
| Male vs | 47 (44.8) | 15 | 32 | 0.68 | 21 | 26 | |
| Female N (%) | 58 (55.2) | 21 | 37 | | 21 | 37 | |
| SBP mean ± SD | 147.7 ± 35.7 | 146.3 ± 44.0 | 146.5 ± 34.9 | 0.94 | 153.0 ± 41.8 | 142.5 ± 35.02 | 0.17 |
| DBP mean ± SD | 82.2 ± 20.1 | 82.9 ± 20.6 | 82.1 ± 32.3 | 0.86 | 85.9 ± 23.1 | 80.0 ± 20.5 | 0.18 |
| HR mean ± SD | 98.9 ± 26.9 | 114.8 ± 28.4 | 96.0 ± 27.1 | 0.001 | 115.1 ± 31.7 | 93.9 ± 23.8 | 0.001 |
| Temperature mean ± SD | 98.4 ± 1.2 | 98.6 ± 1.7 | 98.6 ± 1.2 | 0.72 | 98.6 ± 1.3 | 98.5 ± 1.5 | 0.61 |

| | CXR+ vs CXR− | | P | INT+ vs INT− | | P |
|---|---|---|---|---|---|---|
| N (%) | 35 | 45 | | 18 | 87 | N/A |
| PIRC+ N (%) | 21 | 14 | 0.006 | — | — | |
| PIRC− N (%) | 12 | 31 | | — | — | |
| WBC1; mean ± SD | 12.3 ± 5.4 | 11.1 ± 5.3 | 0.30 | 16.2 ± 7.0 | 9.6 ± 4.3 | 0.001 |
| Hours to WBC1; mean ± SD | 0.9 ± 0.8 | 1.4 ± 1.8 | 0.39 | 1.1 ± 1.1 | 1.1 ± 1.7 | 0.95 |
| WBC2; mean ± SD | 9.6 ± 3.9 | 8.2 ± 3.7 | 0.11 | 12.2 ± 4.1 | 7.6 ± 2.8 | 0.001 |
| Hours to WBC2; mean ± SD | 19.2 ± 9.6 | 16.4 ± 8.7 | 0.18 | 15.9 ± 11.1 | 18.9 ± 9.1 | 0.23 |
| WBC1 <7.9; N (%) | 6 | 14 | 0.16 | 1 | 35 | 0.001 |
| WBC1 7.9-10.5; N (%) | 6 | 11 | | 3 | 22 | |
| WBC1 10.6-13.2; N (%) | 14 | 8 | | 5 | 15 | |
| WBCl 13.3-31.2; N (%) | 9 | 12 | | 9 | 15 | |
| Hemoglobin; mean ± SD | 12.7 ± 2.0 | 13.4 ± 1.9 | 0.11 | 13.1 ± 2.2 | 13.1 ± 1.8 | 0.98 |
| Platelets ×10³; mean ± SD | 223.2 ± 72.3 | 221.6 ± 79.7 | 0.84 | 275.8 ± 88.8 | 207.7 ± 66.4 | 0.001 |
| CXR+ | — | — | — | 12 | 21 | 0.03 |
| CXR− | — | — | | 6 | 39 | |
| Hours to CXR; mean ± SD | 3.6 ± 5.38 | 2.7 ± 4.5 | 0.93 | 1.8 ± 1.4 | 8.1 ± 20.0 | 0.07 |
| Age; years mean ± SD | 64.7 ± 17.3 | 60.6 ± 16.2 | 0.18 | 58.6 ± 13.2 | 61.0 ± 17.9 | 0.51 |
| Male vs | 14 | 23 | 0.37 | 7 | 40 | 0.61 |
| Female N (%) | 22 | 22 | | 11 | 47 | |
| SBP mean ± SD | 143.3 ± 42.0 | 147.7 ± 35.2 | 0.61 | 153.9 ± 51.4 | 145.2 ± 34.9 | 0.54 |
| DBP mean ± SD | 79.1 ± 21.9 | 85.8 ± 22.0 | 0.18 | 89.9 ± 23.4 | 80.8 ± 21.1 | 0.1 |
| HR mean ± SD | 105.3 ± 29.0 | 102.9 ± 28.3 | 0.71 | 114.6 ± 34.7 | 99.9 ± 27.0 | 0.05 |
| Temperature mean ± SD | 98.8 ± 1.9 | 98.4 ± 1.1 | 0.19 | 98.6 ± 2.2 | 98.5 ± 1.2 | 0.28 |

PeCRC+ = Periconvulsive respiratory compromise; PeCRC− = Absence of periconvulsive respiratory compromise; PIL+ = Postictal leukocytosis, defined as WBC1 count ≥11,000 cells/mm³; PIL− = postictal WSC1 count <11.000 cells/mm³; CXR+ = Abnormal chest X ray; CXR− = Normal chest X ray; INT+ = Intubated; INT− = Not intubated; Min. = minutes; SBP = Systolic blood pressure; DBP = Diastolic blood pressure; WBC1 = Initial postictal white blood cell count; WBC2 = Second WBC measured within 36 h of WBC1.

TABLE 8
ROC table for WBC1 as a predictor of intubation

| WBC1 | | | | | |
|---|---|---|---|---|---|
| (10³ cells/ | | | | | Likelihood |
| mm³) | Sensitivity % | 95% CI | Specificity % | 95% CI | Ratio |
| <3.400 | 0.7634 | 0.03916% to 4.197% | 100.0 | 84.54% to 100.0% | |
| <3.650 | 1.527 | 0.2713% to 5.396% | 100.0 | 84.54% to 100.0% | |
| <3.900 | 2.290 | 0.6242% to 6.517% | 100.0 | 84.54% to 100.0% | |
| <4.150 | 3.817 | 1.641% to 8.624% | 100.0 | 84.54% to 100.0% | |
| <4.350 | 5.344 | 2.612% to 10.62% | 100.0 | 84.54% to 100.0% | |
| <4.550 | 6.870 | 3.656% to 12.54% | 100.0 | 84.54% to 100.0% | |
| <4.750 | 8.397 | 4.753% to 14.41% | 100.0 | 84.54% to 100.0% | |
| <4.850 | 9.160 | 5.318% to 15.33% | 100.0 | 84.54% to 100.0% | |
| <4.950 | 9.924 | 5.891% to 16.24% | 100.0 | 84.54% to 100.0% | |
| <5.050 | 10.69 | 6.473% to 17.14% | 95.24 | 77.33% to 99.76% | 2.244 |
| <5.250 | 12.21 | 7.660% to 18.92% | 95.24 | 77.33% to 99.76% | 2.565 |
| <5.450 | 12.98 | 8.263% to 19.80% | 95.24 | 77.33% to 99.76% | 2.725 |
| <5.600 | 13.74 | 8.871% to 20.68% | 95.24 | 77.33% to 99.76% | 2.885 |
| <5.750 | 16.79 | 11.36% to 24.12% | 95.24 | 77.33% to 99.76% | 3.527 |
| <5.850 | 17.56 | 11.99% to 24.97% | 95.24 | 77.33% to 99.76% | 3.687 |
| <5.950 | 18.32 | 12.63% to 25.81% | 95.24 | 77.33% to 99.76% | 3.847 |
| <6.050 | 19.08 | 13.27% to 26.66% | 95.24 | 77.33% to 99.76% | 4.008 |
| <6.150 | 22.14 | 15.88% to 29.98% | 95.24 | 77.33% to 99.76% | 4.649 |
| <6.250 | 25.95 | 19.21% to 34.07% | 95.24 | 77.33% to 99.76% | 5.450 |
| <6.350 | 27.48 | 20.56% to 35.68% | 95.24 | 77.33% to 99.76% | 5.771 |
| <6.450 | 30.53 | 23.30% to 38.88% | 95.24 | 77.33% to 99.76% | 6.412 |
| <6.550 | 31.30 | 23.99% to 39.68% | 95.24 | 77.33% to 99.76% | 6.573 |
| <6.750 | 32.06 | 24.68% to 40.47% | 95.24 | 77.33% to 99.76% | 6.733 |
| <6.950 | 32.82 | 25.37% to 41.25% | 95.24 | 77.33% to 99.76% | 6.893 |
| <7.050 | 35.88 | 28.17% to 44.39% | 95.24 | 77.33% to 99.76% | 7.534 |
| <7.150 | 36.64 | 28.88% to 45.16% | 95.24 | 77.33% to 99.76% | 7.695 |
| <7.250 | 38.17 | 30.30% to 46.71% | 95.24 | 77.33% to 99.76% | 8.015 |
| <7.350 | 39.69 | 31.72% to 48.25% | 95.24 | 77.33% to 99.76% | 8.336 |
| <7.450 | 40.46 | 32.44% to 49.02% | 95.24 | 77.33% to 99.76% | 8.496 |
| <7.550 | 41.98 | 33.88% to 50.55% | 95.24 | 77.33% to 99.76% | 8.817 |
| <7.650 | 43.51 | 35.33% to 52.07% | 95.24 | 77.33% to 99.76% | 9.137 |
| <7.750 | 44.27 | 36.05% to 52.82% | 95.24 | 77.33% to 99.76% | 9.298 |
| <7.850 | 45.04 | 36.78% to 53.58% | 95.24 | 77.33% to 99.76% | 9.458 |
| <7.950 | 47.33 | 38.98% to 55.83% | 95.24 | 77.33% to 99.76% | 9.939 |
| <8.050 | 48.09 | 39.71% to 56.58% | 95.24 | 77.33% to 99.76% | 10.10 |
| <8.200 | 48.85 | 40.45% to 57.32% | 95.24 | 77.33% to 99.76% | 10.26 |
| <8.400 | 51.15 | 42.68% to 59.55% | 95.24 | 77.33% to 99.76% | 10.74 |
| <8.550 | 51.91 | 43.42% to 60.29% | 95.24 | 77.33% to 99.76% | 10.90 |
| <8.650 | 53.44 | 44.92% to 61.76% | 95.24 | 77.33% to 99.76% | 11.22 |
| <8.750 | 54.96 | 46.42% to 63.22% | 95.24 | 77.33% to 99.76% | 11.54 |
| <8.850 | 55.73 | 47.18% to 63.95% | 95.24 | 77.33% to 99.76% | 11.70 |
| <8.950 | 56.49 | 47.93% to 64.67% | 95.24 | 77.33% to 99.76% | 11.86 |
| <9.050 | 57.25 | 48.69% to 65.40% | 95.24 | 77.33% to 99.76% | 12.02 |
| <9.200 | 58.02 | 49.45% to 66.12% | 95.24 | 77.33% to 99.76% | 12.18 |
| <9.350 | 59.54 | 50.98% to 67.56% | 90.48 | 71.09% to 98.31% | 6.252 |
| <9.500 | 60.31 | 51.75% to 68.28% | 85.71 | 65.36% to 95.02% | 4.221 |
| <9.700 | 61.07 | 52.52% to 68.99% | 85.71 | 65.36% to 95.02% | 4.275 |
| <9.900 | 62.60 | 54.06% to 70.41% | 85.71 | 65.36% to 95.02% | 4.382 |
| <10.05 | 64.12 | 55.61% to 71.83% | 85.71 | 65.36% to 95.02% | 4.489 |
| <10.15 | 64.89 | 56.39% to 72.53% | 85.71 | 65.36% to 95.02% | 4.542 |
| <10.35 | 66.41 | 57.96% to 73.93% | 85.71 | 65.36% to 95.02% | 4.649 |
| <10.55 | 66.41 | 57.96% to 73.93% | 80.95 | 60.06% to 92.33% | 3.487 |
| <10.65 | 67.18 | 58.75% to 74.63% | 80.95 | 60.00% to 92.33% | 3.527 |
| <10.75 | 67.18 | 58.75% to 74.63% | 76.19 | 54.91% to 89.37% | 2.821 |
| <10.90 | 68.70 | 60.32% to 76.01% | 76.19 | 54.91% to 89.37% | 2.885 |
| <11.05 | 70.23 | 61.91% to 77.39% | 76.19 | 54.91% to 89.37% | 2.950 |
| <11.15 | 71.76 | 63.51% to 78.76% | 71.43 | 50.04% to 86.19% | 2.511 |
| <11.25 | 73.28 | 65.12% to 80.12% | 71.43 | 50.04% to 86.19% | 2.565 |
| <11.35 | 74.05 | 65.93% to 80.79% | 71.43 | 50.04% to 86.19% | 2.592 |
| <11.45 | 75.57 | 67.56% to 82.13% | 71.43 | 50.04% to 86.19% | 2.645 |
| <11.55 | 76.34 | 68.37% to 82.80% | 71.43 | 50.04% to 86.19% | 2.672 |
| <11.65 | 77.10 | 69.19% to 83.46% | 71.43 | 50.04% to 86.19% | 2.698 |
| <11.80 | 77.86 | 70.02% to 84.12% | 66.67 | 45.37% to 82.81% | 2.336 |
| <12.05 | 78.63 | 70.84% to 84.78% | 61.90 | 40.88% to 79.25% | 2.064 |
| <12.40 | 79.39 | 71.67% to 85.43% | 61.90 | 40.88% to 79.25% | 2.084 |
| <12.65 | 81.68 | 74.19% to 87.37% | 61.90 | 40.88% to 79.25% | 2.144 |
| <12.75 | 83.21 | 75.88% to 88.64% | 61.90 | 40.88% to 79.25% | 2.184 |
| <12.85 | 83.21 | 75.88% to 88.64% | 52.38 | 32.37% to 71.66% | 1.747 |
| <12.95 | 83.97 | 76.73% to 89.27% | 52.38 | 32.37% to 71.66% | 1.763 |
| <13.10 | 83.97 | 76.73% to 89.27% | 47.62 | 28.34% to 67.63% | 1.603 |
| <13.25 | 84.73 | 77.59% to 89.89% | 47.62 | 28.34% to 67.63% | 1.618 |
| <13.55 | 85.50 | 78.46% to 90.51% | 47.62 | 28.34% to 67.63% | 1.632 |
| <13.85 | 86.26 | 79.32% to 91.13% | 47.62 | 28.34% to 67.63% | 1.647 |
| <14.05 | 87.02 | 80.20% to 91.74% | 47.62 | 28.34% to 67.63% | 1.661 |
| <14.25 | 87.79 | 81.08% to 92.34% | 47.62 | 28.34% to 67.63% | 1.676 |
| <14.60 | 88.55 | 81.97% to 92.94% | 47.62 | 28.34% to 67.63% | 1.690 |
| <14.95 | 89.31 | 82.86% to 93.53% | 47.62 | 28.34% to 67.63% | 1.705 |
| <15.10 | 90.08 | 83.76% to 94.11% | 47.62 | 28.34% to 67.63% | 1.720 |
| <15.30 | 90.08 | 83.76% to 94.11% | 42.86 | 24.47% to 63.45% | 1.576 |
| <15.65 | 90.08 | 83.76% to 94.11% | 38.10 | 20.75% to 59.12% | 1.455 |
| <15.95 | 90.84 | 84.67% to 94.68% | 38.10 | 20.75% to 59.12% | 1.467 |
| <16.05 | 91.60 | 85.59% to 95.25% | 38.10 | 20.75% to 59.12% | 1.480 |
| <16.30 | 91.60 | 85.59% to 95.25% | 33.33 | 17.19% to 54.63% | 1.374 |
| <16.60 | 92.37 | 86.52% to 95.80% | 28.57 | 13.81% to 49.96% | 1.293 |
| <16.80 | 93.13 | 87.46% to 96.34% | 28.57 | 13.81% to 49.96% | 1.304 |
| <17.15 | 93.89 | 88.41% to 96.87% | 28.57 | 13.81% to 49.96% | 1.315 |
| <17.65 | 94.66 | 89.38% to 97.39% | 28.57 | 13.81% to 49.96% | 1.325 |
| <17.95 | 95.42 | 90.37% to 97.88% | 28.57 | 13.81% to 49.96% | 1.336 |
| <18.05 | 96.18 | 91.38% to 98.36% | 28.57 | 13.81% to 49.96% | 1.347 |
| <18.45 | 96.95 | 92.41% to 98.81% | 28.57 | 13.81% to 49.96% | 1.357 |
| <19.10 | 97.71 | 93.48% to 99.38% | 28.57 | 13.81% to 49.96% | 1.368 |
| <19.85 | 98.47 | 94.60% to 99.73% | 28.57 | 13.81% to 49.96% | 1.379 |
| <21.10 | 99.24 | 95.80% to 99.96% | 28.57 | 13.81% to 49.96% | 1.389 |
| <21.95 | 99.24 | 95.80% to 99.96% | 19.05 | 7.668% to 40.00% | 1.226 |
| <22.40 | 99.24 | 95.80% to 99.96% | 14.29 | 4.981% to 34.64% | 1.158 |
| <23.60 | 99.24 | 95.80% to 99.96% | 9.524 | 1.692% to 28.91% | 1.097 |
| <26.20 | 100.0 | 97.15% to 100.0% | 9.524 | 1.692% to 28.91% | 1.105 |
| <29.60 | 100.0 | 97.15% to 100.0% | 4.762 | 0.2443% to 22.67% | 1.050 |
| <3.400 | 0.7634 | 0.03916% to 4.197% | 100.0 | 84.54% to 100.0% | |

TABLE 9
ROC table for initial triage heart rate as a predictor of intubation

| Heart Rate | | | | | |
|---|---|---|---|---|---|
| (beats per | | | | | Likelihood |
| minute) | Sensitivity % | 95% CI | Specificity % | 95% CI | Ratio |
| <51.5 | 0 | 0.00% to 2.85% | 95.2 | 77.3% to 99.8% | 0 |
| <55.0 | 0.763 | 0.0392% to 4.20% | 95.2 | 77.3% to 99.8% | 0.16 |
| <57.5 | 2.29 | 0.624% to 6.52% | 95.2 | 77.3% to 99.8% | 0.481 |
| <59.0 | 3.82 | 1.64% to 8.62% | 95.2 | 77.3% to 99.8% | 0.802 |
| <61.0 | 5.34 | 2.61% to 10.6% | 95.2 | 77.3% to 99.8% | 1.12 |
| <63.0 | 6.11 | 3.13% to 11.6% | 95.2 | 77.3% to 99.8% | 1.28 |
| <66.5 | 7.63 | 4.20% to 13.5% | 95.2 | 77.3% to 99.8% | 1.6 |
| <69.5 | 9.16 | 5.32% to 15.3% | 90.5 | 71.1% to 98.3% | 0.962 |
| <70.5 | 10.7 | 6.47% to 17.1% | 85.7 | 65.4% to 95.0% | 0.748 |
| <71.5 | 13 | 8.26% to 19.8% | 85.7 | 65.4% to 95.0% | 0.908 |
| <73.0 | 16 | 10.7% to 23.3% | 81 | 60.0% to 92.3% | 0.842 |
| <74.5 | 18.3 | 12.6% to 25.8% | 81 | 60.0% to 92.3% | 0.962 |
| <75.5 | 19.1 | 13.3% to 26.7% | 81 | 60.0% to 92.3% | 1 |
| <76.5 | 21.4 | 15.2% to 29.2% | 81 | 60.0% to 92.3% | 1.12 |
| <77.5 | 22.1 | 15.9% to 30.0% | 81 | 60.0% to 92.3% | 1.16 |
| <79.0 | 24.4 | 17.9% to 32.4% | 81 | 60.0% to 92.3% | 1.28 |
| <81.0 | 26.7 | 19.9% to 34.9% | 76.2 | 54.9% to 89.4% | 1.12 |
| <82.5 | 28.2 | 21.2% to 36.5% | 76.2 | 54.9% to 89.4% | 1.19 |
| <83.5 | 29.8 | 22.6% to 38.1% | 76.2 | 54.9% to 89.4% | 1.25 |
| <84.5 | 34.4 | 26.8% to 42.8% | 76.2 | 54.9% to 89.4% | 1.44 |
| <85.5 | 36.6 | 28.9% to 45.2% | 76.2 | 54.9% to 89.4% | 1.54 |
| <86.5 | 38.9 | 31.0% to 47.5% | 76.2 | 54.9% to 89.4% | 1.64 |
| <87.5 | 40.5 | 32.4% to 49.0% | 76.2 | 54.9% to 89.4% | 1.7 |
| <88.5 | 4.2 | 33.9% to 50.5% | 76.2 | 54.9% to 89.4% | 1.76 |
| <89.5 | 43.5 | 35.3% to 52.1% | 76.2 | 54.9% to 89.4% | 1.83 |
| <90.5 | 45.8 | 37.5% to 54.3% | 76.2 | 54.9% to 89.4% | 1.92 |
| <91.5 | 46.6 | 38.2% to 55.1% | 76.2 | 54.9% to 89.4% | 1.96 |
| <92.5 | 48.9 | 40.5% to 57.3% | 76.2 | 54.9% to 89.4% | 2.05 |
| <93.5 | 51.1 | 42.7% to 59.5% | 76.2 | 54.9% to 89.4% | 2.15 |
| <95.0 | 53.4 | 44.9% to 61.8% | 71.4 | 50.0% to 86.2% | 1.87 |
| <96.5 | 55 | 46.4% to 63.2% | 66.7 | 45.4% to 82.8% | 1.65 |
| <97.5 | 55.7 | 47.2% to 63.9% | 66.7 | 45.4% to 82.8% | 1.67 |
| <98.5 | 58.8 | 50.2% to 66.8% | 66.7 | 45.4% to 82.8% | 1.76 |
| <99.5 | 60.3 | 51.7% to 68.3% | 66.7 | 45.4% to 82.8% | 1.81 |
| <101 | 62.6 | 54.1% to 70.4% | 57.1 | 36.5% to 75.5% | 1.46 |
| <102 | 63.4 | 54.8% to 71.1% | 57.1 | 36.5% to 75.5% | 1.48 |
| <103 | 65.6 | 57.2% to 73.2% | 57.1 | 36.5% to 75.5% | 1.53 |
| <104 | 67.9 | 59.5% to 75.3% | 57.1 | 36.5% to 75.5% | 1.59 |
| <105 | 68.7 | 60.3% to 76.0% | 57.1 | 36.5% to 75.5% | 1.6 |
| <106 | 69.5 | 61.1% to 76.7% | 57.1 | 36.5% to 75.5% | 1.62 |
| <107 | 71.8 | 63.5% to 78.8% | 57.1 | 36.5% to 75.5% | 1.67 |
| <108 | 71.8 | 63.5% to 78.8% | 52.4 | 32.4% to 71.7% | 1.51 |
| <109 | 72.5 | 64.3% to 79.4% | 52.4 | 32.4% to 71.7% | 1.52 |
| <110 | 73.3 | 65.1% to 80.1% | 52.4 | 32.4% to 71.7% | 1.54 |
| <111 | 75.6 | 67.6% to 82.1% | 42.9 | 24.5% to 63.5% | 1.32 |
| <113 | 78.6 | 70.8% to 84.8% | 42.9 | 24.5% to 63.5% | 1.38 |
| <115 | 80.2 | 72.5% to 86.1% | 42.9 | 24.5% to 63.5% | 1.4 |
| <117 | 80.9 | 73.3% to 86.7% | 42.9 | 24.5% to 63.5% | 1.42 |
| <118 | 81.7 | 74.2% to 87.4% | 42.9 | 24.5% to 63.5% | 1.43 |
| <119 | 82.4 | 75.0% to 88.0% | 38.1 | 20.8% to 59.1% | 1.33 |
| <121 | 83.2 | 75.9% to 88.6% | 38.1 | 20.8% to 59.1% | 1.34 |
| <123 | 83.2 | 75.9% to 88.6% | 33.3 | 17.2% to 54.6% | 1.25 |
| <124 | 84 | 76.7% to 89.3% | 33.3 | 17.2% to 54.6% | 1.26 |
| <125 | 85.5 | 78.5% to 90.5% | 33.3 | 17.2% to 54.6% | 1.28 |
| <127 | 87 | 80.2% to 91.7% | 28.6 | 13.8% to 50.0% | 1.22 |
| <129 | 87.8 | 81.1% to 92.3% | 28.6 | 13.8% to 50.0% | 1.23 |
| <131 | 88.5 | 82.0% to 92.9% | 28.6 | 13.8% to 50.0% | 1.24 |
| <132 | 89.3 | 82.9% to 93.5% | 28.6 | 13.8% to 50.0% | 1.25 |
| <133 | 90.1 | 83.8% to 94.1% | 28.6 | 13.8% to 50.0% | 1.26 |
| <135 | 91.6 | 85.6% to 95.2% | 28.6 | 13.8% to 50.0% | 1.28 |
| <138 | 93.1 | 87.5% to 96.3% | 23.8 | 10.6% to 45.1% | 1.22 |
| <141 | 93.9 | 88.4% to 96.9% | 23.8 | 10.6% to 45.1% | 1.23 |
| <143 | 94.7 | 89.4% to 97.4% | 19 | 7.67% to 40.0% | 1.17 |
| <145 | 95.4 | 90.4% to 97.9% | 19 | 7.67% to 40.0% | 1.18 |
| <147 | 95.4 | 90.4% to 97.9% | 14.3 | 4.98% to 34.6% | 1.11 |
| <150 | 96.9 | 92.4% to 98.8% | 14.3 | 4.98% to 34.6% | 1.13 |
| <153 | 97.7 | 93.5% to 99.4% | 14.3 | 4.98% to 34.6% | 1.14 |
| <155 | 98.5 | 94.6% to 99.7% | 14.3 | 4.98% to 34.6% | 1.15 |
| <157 | 98.5 | 94.6% to 99.7% | 9.52 | 1.69% to 28.9% | 1.09 |
| <162 | 99.2 | 95.8% to 100% | 9.52 | 1.69% to 28.9% | 1.1 |
| <170 | 99.2 | 95.8% to 100% | 4.76 | 0.244% to 22.7% | 1.04 |
| <190 | 99.2 | 95.8% to 100% | 0 | 0.00% to 15.5% | 0.992 |
| <51.5 | 0 | 0.00% to 2.85% | 95.2 | 77.3% to 99.8% | 0 |

1. A method for determining the severity of a mammalian dive response (MDR) triggered during a patient's seizure comprising the following steps:
(1) collecting an ictal specimen, the ictal specimen being a saliva or any other secretion from the nose or mouth of the patient when the patient is in a peri-ictal period;
(2) analyzing the ictal specimen to measure a level of a specimen marker, the specimen marker being a cellular or non-cellular biological component; and
(3) determining the severity of the MDR based on the level of the specimen marker.

2. The method of claim 1, wherein step (2) comprises
collecting a reference specimen, the reference specimen being a saliva or any other secretion from the nose or mouth of the patient when the patient is not having a seizure or from a pool of healthy age-appropriate seizure-free volunteers; and
analyzing the ictal specimen to measure the level of the specimen marker;
analyzing the reference specimen to measure a level of the specimen marker; and
comparing the level of the specimen marker in the ictal specimen with the level of the specimen marker in the reference specimen.

3. The method of claim 2, wherein the ictal specimen and the reference specimen are a tissue, a fluid, a cell, an electrolyte, or a molecule found in or around a respiratory system.

4. The method of claim 3, wherein the ictal specimen and reference specimen are a cell that makes up an oro-nasal cavity, a pharyngeal cavity, or a lung parenchyma; a cell that makes up lung blood vessels; a molecule that makes up connective tissues of lungs and tracheobronchial tree; an electrolyte or a cell or platelet that circulates throughout a lung, peritracheal, peri-bronchial arteries, veins or lymphatic vessels; or an electrolyte or a molecule, cell or fluid that makes up or that extravasates into lung alveoli, or into the lumen of tracheobronchial tree.

5. The method of claim 2, wherein the ictal specimen and reference specimen are a biological component that enters a lung through pulmonary arteries.

6. The method of claim 1, wherein the specimen marker is selected from the group consisting of electrolytes, carbohydrates, proteins, lipids, and nucleic acids; or the specimen marker is a surfactant.

7. (canceled)

8. The method of claim 2, wherein step (3) comprises:
determining that the severity of the MDR is high and a prophylactic pharmacological approach is required when the level of the specimen marker in the ictal specimen is 2 times, 5 times, or 10 times higher than the level of the specimen marker in the reference specimen.

9. The method of claim 8, wherein the prophylactic pharmacological approach is to administer to the patient an adrenergic blocking agent selected from the group consisting of n-g(fluorenyl)-n ethyl-β chloroethylamine (SKF-501), Phentolamine, Doxazosin, Terazosin, Tamsulosin, Alfuzosin, Phenoxybenzaamine, Yohimbine, Imipramine, Silodosin, Amitriptilyne, Indoramine, Abanoquil, Corynanthine, Ajmalicine, Quetiapine, Dapiprazone, Amoxapine, Risperidone, Idazoxan, Bunazosin, Doxepin, Atiprosin, Piperoxan, Fenmetozole, Denopamine, Dobutamine, Dopexamine, Epinephrine, Isoprenaline, Isoproterenol, Prenalterol, Xamotero, Arformoterol, Buphenine, Clenbuterol, Dopexamine, Fenoterol, Formoterol, Isoetarine, Levosalbutamol, levalbuterol, Orciprenaline, metaproterenol, Pirbuterol, Procaterol, Ritodrine, Salbutamol, albuterol, Salmeterol, Terbutaline, Arbutamine, Befunolol, Bromoacetylalprenololmenthane, Broxaterol, Cimaterol, Cirazoline, Etilefrine, Hexoprenaline, Higenamine, Isoxsuprine, Mabuterol, Methoxyphenamine, Oxyfedrine, Ractopamine, Reproterol, Rimiterol, Tretoquinol, Tulobuterol, Zilpaterol, Zinterol, Propranolol, Bucindolol, Carteolol, Carvedilol, Labetalol, Nadolol, Oxprenolol, Penbutolol, Pindolol, Sotalol, Timolol, Acebutolol, Atenolol, Betaxolol, Bisoprolol, Celiprolol, Metoprolol, Nebivolol, Esmolol, Butaxamine, ICI-118,551, SR 59230A, 4-NEMD, 7-Me-marsanidine, Agmatine, Apraclonidine, Brimonidine, Cannabigerol, Clonidine, Detomidine, Dexmedetomidine, Fadolmidine, Guanabenz, Guanfacine, Lofexidine, Marsanidine, Medetomidine, Methamphetamine, Mivazerol, Rilmenidine, Romifidine, Talipexole, Tiamenidine, Tizanidine, Tolonidine, Xylazine, Xylometazoline, L-748,328, L-748,337 and SR 59,230A, Amibegron (SR-58611A), CL-316,243, L-742,791, L-796,568, LY-368,842, Mirabegron (YM-178), Ro40-2148, Solabegron (GW-427,353), and Vibegron (MK-4618); or to administer to the patient a sympathetic ganglionic blocking agent selected from the group consisting of hexamethonium, pentolinium, mecamylamine, trimetaphan, tubocurarine, pempidine, benzohexonium, chlorisondamine, pentamine, and tetra ethylammonium chloride; or to administer to the patient a cholinergic receptor blocking agent selected from the group consisting of atropine, scopolamine, homatropine, tropicamide, benzproine, biperiden, trihexyphenidyl, oxybutynin, tolterodine, solifenacin, dicyclomine, darifenacin, glycopyrrolate, ipratropium, and tiotropium; or to administer the patient with a combination thereof.

10. The method of claim 1, wherein step (2) comprises an analysis selected from a group consisting of mass spectrometry, protein profile analysis, protein chip array analysis, lipid profile analysis, nucleic acid profile analysis, and immunofluorescence.

11. The method of claim 1, wherein the ictal specimen and the reference specimen are measured by a portable sample analyzing unit.

12. A method for determining the severity of a mammalian dive response (MDR) or similar autonomic reflex triggered during a seizure comprising the following steps:
(1) collecting an ictal blood sample from a patient when the patient is in a peri-ictal period;
(2) analyzing the ictal blood sample for the levels of one or more blood markers;
(3) collecting a reference blood sample from the patient when the patient is not having a seizure;
(4) analyzing the reference blood sample for the levels of the one or more blood markers; and
(5) comparing the levels of the one or more blood markers in the ictal blood sample and the levels of the one or more blood markers in the reference blood sample.

13. The method of claim 12, wherein the one or more blood markers are selected from the group consisting of electrolytes, carbohydrates, proteins, lipids, nucleic acids, cell surface molecules, a human body tissue (e.g., lungs, muscles, liver, spleen, intestines, bone marrow, arteries, veins, etc.), white blood cells, red blood cells, platelets, pH, lactic acid level, carbon dioxide level, oxygen level, and blood oxygen saturation level.

14. The method of claim 12, further comprising:
measuring the patient's pulse.

15. The method of claim 13, further comprising:
determining that the severity of the MDR triggered during a seizure is severe and a prophylactic pharmacological approach is required when the levels of the one or more blood markers in the ictal blood sample are 2 times, 5 times, or 10 times than the levels of the one or more blood markers in the reference blood sample.

16. The method of claim 13, further comprising:
determining that the severity of the MDR triggered during a seizure is severe and a prophylactic pharmacological approach is required when the levels of the one or more blood markers in the ictal blood sample are 3%, 5%, or 10% lower than the levels of the one or more blood markers in the reference blood sample, wherein the one or more blood markers are pH or oxygen level.

17. The method of claim 12, further comprising:
determining that the severity of the MDR triggered during a seizure is high and a prophylactic pharmacological approach is required when the blood white cell count in the ictal blood sample is higher than 9,200 cells/mm³, 16,000 cells/mm³, 18,000 cells/mm³, 20,000 cells/mm³, 22,000 cells/mm³, 24,000 cells/mm³, 26,000 cells/mm³, 28,000 cells/mm³, 30,000 cells/mm³, or 32,000 cells/mm³, wherein the one or more blood markers are blood white cell.

18. The method of claim 12, wherein the ictal blood sample and reference blood sample are measured by a portable sample analyzing unit.

19. The method of claim 15, wherein the prophylactic pharmacological approach is to administer to the patient an adrenergic blocking agent selected from the group consisting of n-g(fluorenyl)-n ethyl-β chloroethylamine (SKF-501), Phentolamine, Doxazosin, Terazosin, Tamsulosin, Alfuzosin, Phenoxybenzaamine, Yohimbine, Imipramine, Silodosin, Amitriptilyne, Indoramine, Abanoquil, Corynanthine, Ajmalicine, Quetiapine, Dapiprazone, Amoxapine, Risperidone, Idazoxan, Bunazosin, Doxepin, Atiprosin, Piperoxan, Fenmetozole, Denopamine, Dobutamine, Dopexamine, Epinephrine, Isoprenaline, Isoproterenol, Prenalterol, Xamotero, Arformoterol, Buphenine, Clenbuterol, Dopexamine, Fenoterol, Formoterol, Isoetarine, Levosalbutamol, levalbuterol, Orciprenaline, metaproterenol, Pirbuterol, Procaterol, Ritodrine, Salbutamol, albuterol, Salmeterol, Terbutaline, Arbutamine, Befunolol, Bromoacetylalprenololmenthane, Broxaterol, Cimaterol, Cirazoline, Etilefrine, Hexoprenaline, Higenamine, Isoxsuprine, Mabuterol, Methoxyphenamine, Oxyfedrine, Ractopamine, Reproterol, Rimiterol, Tretoquinol, Tulobuterol, Zilpaterol, Zinterol, Propranolol, Bucindolol, Carteolol, Carvedilol, Labetalol, Nadolol, Oxprenolol, Penbutolol, Pindolol, Sotalol, Timolol, Acebutolol, Atenolol, Betaxolol, Bisoprolol, Celiprolol, Metoprolol, Nebivolol, Esmolol, Butaxamine, ICI-118,551, SR 59230A, 4-NEMD, 7-Me-marsanidine, Agmatine, Apraclonidine, Brimonidine, Cannabigerol, Clonidine, Detomidine, Dexmedetomidine, Fadolmidine, Guanabenz, Guanfacine, Lofexidine, Marsanidine, Medetomidine, Methamphetamine, Mivazerol, Rilmenidine, Romifidine, Talipexole, Tiamenidine, Tizanidine, Tolonidine, Xylazine, Xylometazoline, L-748,328, L-748,337 and SR 59,230A, Amibegron (SR-58611A), CL-316,243, L-742,791, L-796,568, LY-368,842, Mirabegron (YM-178), Ro40-2148, Solabegron (GW-427,353), and Vibegron (MK-4618); or to administer to the patient a sympathetic ganglionic blocking agent selected from the group consisting of hexamethonium, pentolinium, mecamylamine, trimetaphan, tubocurarine, pempidine, benzohexonium, chlorisondamine, pentamine, and tetra ethylammonium chloride; or to administer to the patient a cholinergic receptor blocking agent selected from the group consisting of atropine, scopolamine, homatropine, tropicamide, benzproine, biperiden, trihexyphenidyl, oxybutynin, tolterodine, solifenacin, dicyclomine, darifenacin, glycopyrrolate, ipratropium, and tiotropium; or to administer the patient with a combination thereof.

20. A method for treating epileptic seizures comprising:
administering to a patient a combination of an adrenergic blocking agent and an anti-seizure agent; or a combination of a cholinergic blocking and an anti-seizure agent.

21. The method of claim 20, wherein:
the adrenergic blocking agent is selected from the group consisting of n-g(fluorenyl)-n ethyl-β chloroethylamine (SKF-501), Phentolamine, Doxazosin, Terazosin, Tamsulosin, Alfuzosin, Phenoxybenzaamine, Yohimbine, Imipramine, Silodosin, Amitriptilyne, Indoramine, Abanoquil, Corynanthine, Ajmalicine, Quetiapine, Dapiprazone, Amoxapine, Risperidone, Idazoxan, Bunazosin, Doxepin, Atiprosin, Piperoxan, Fenmetozole, Denopamine, Dobutamine, Dopexamine, Epinephrine, Isoprenaline, Isoproterenol, Prenalterol, Xamotero, Arformoterol, Buphenine, Clenbuterol, Dopexamine, Epinephrine, Fenoterol, Formoterol, Isoetarine, Levosalbutamol, levalbuterol, Orciprenaline, metaproterenol, Pirbuterol, Procaterol, Ritodrine, Salbutamol, albuterol, Salmeterol, Terbutaline, Arbutamine, Befunolol, Bromoacetylalprenololmenthane, Broxaterol, Cimaterol, Cirazoline, Etilefrine, Hexoprenaline, Higenamine, Isoxsuprine, Mabuterol, Methoxyphenamine, Oxyfedrine, Ractopamine, Reproterol, Rimiterol, Tretoquinol, Tulobuterol, Zilpaterol, Zinterol, Propranolol, Bucindolol, Carteolol, Carvedilol, Labetalol, Nadolol, Oxprenolol, Penbutolol, Pindolol, Sotalol, Timolol, Acebutolol, Atenolol, Betaxolol, Bisoprolol, Celiprolol, Metoprolol, Nebivolol, Esmolol, Butaxamine, ICI-118,551, SR 59230A, 4-NEMD, 7-Me-marsanidine, Agmatine, Apraclonidine, Brimonidine, Cannabigerol, Clonidine, Detomidine, Dexmedetomidine, Fadolmidine, Guanabenz, Guanfacine, Lofexidine, Marsanidine, Medetomidine, Methamphetamine, Mivazerol, Rilmenidine, Romifidine, Talipexole, Tiamenidine, Tizanidine, Tolonidine, Xylazine, Xylometazoline, L-748,328, L-748,337 and SR 59,230A, Amibegron (SR-58611A), CL-316,243, L-742,791, L-796,568, LY-368,842, Mirabegron (YM-178), Ro40-2148, Solabegron (GW-427,353), Vibegron (MK-4618), hexamethonium, pentolinium, mecamylamine, trimetaphan, tubocurarine, pempidine, benzohexonium, chlorisondamine, and pentaamine;
the cholinergic blocking agent is selected from the group consisting of atropine, scopolamine, homatropine, tropicamide, benzproine, biperiden, trihexyphenidyl, oxybutynin, tolterodine, solifenacin, dicyclomine, darifenacin, glycopyrrolate, ipratropium, and tiotropium; and
the anti-seizure agent is selected from the group consisting of phenytoin, carbamazepine, clobazam, divalproex, eslicarbazepine, oxcarbazepine, lacosamide, ethosuximide, gabapentin, methsuxamide, perampanel, phenobarbital, pregabalin, rufinamide, tiagabine, vigabatrin, levetiracetam, clonazepam, clorazepate, ezogabine, felbamate, lamotrigine, lorazepam, primidone, topiramate, valproic acid, and zonisamide.
